(19) United States
(12) Patent Application Publication
(10) Pub. No.: US 2013/0122043 A1
(43) Pub. Date: May 16, 2013

(54) MODIFIED POLYPEPTIDES AND PROTEINS AND USES THEREOF

(71) Applicants: Carla Guimaraes, Boston, MA (US); Hidde Ploegh, Brookline, MA (US)

(72) Inventors: Carla Guimaraes, Boston, MA (US); Hidde Ploegh, Brookline, MA (US)

(73) Assignee: WHITEHEAD INSTITUTE FOR BIOMEDICAL RESEARCH, Cambridge, MA (US)

(21) Appl. No.: 13/642,458

(22) PCT Filed: Apr. 20, 2011
(86) PCT No.: PCT/US11/33303

§ 371 Date: Feb. 1, 2013

Related U.S. Application Data
(60) Provisional application No. 61/326,080, filed on Apr. 20, 2010.

(51) Int. Cl. C07K 14/00; C12P 21/00; C07K 2/00

(52) U.S. Cl. CPC C07K 14/00; C07K 2/00; C12P 21/00
USPC 424/204.1; 530/300; 435/68.1; 530/350; 530/322; 530/395; 435/375; 424/236.1

(57) ABSTRACT

The present invention provides modified multi-chain and multi-subunit proteins and methods for making them. In some protease embodiments the proteins are modified AB₅ toxins in which a compound of interest is attached to the A1 chain.

### RELATED APPLICATIONS

This application claims the benefit of, and priority to, U.S. provisional application Ser. No. 61/326,080, filed Apr. 20, 2010, the entire content of which is incorporated by reference herein.

### BACKGROUND OF THE INVENTION

Site-specific labeling and conjugation of proteins are of fundamental importance in protein engineering. While a variety of techniques for site-specific modification of polypeptides are available, advances in this area are of great interest.

### SUMMARY OF THE INVENTION

The present invention relates to compositions and methods useful for site-specific modification of proteolytically processed polypeptides and multi-chain proteins that contain at least one proteolytically processed polypeptide. In some aspects, the invention relates to engineered polypeptides that are substrates for transamidase-catalyzed ligation of a compound of interest thereto. The invention also relates to multi-chain and multi-subunit proteins that contain at least one modified proteolytically processed polypeptide. In some embodiments, the multi-chain polypeptide is a subunit of a bacterial exotoxin, e.g., an ABₙ toxin, e.g., an AB₅ toxin such as cholera toxin. In some aspects, the invention relates to a modified bacterial AB₅ toxin that has a compound of interest attached to the A1 chain. In some embodiments the compound of interest is attached at or near the C-terminus of the A1 chain. The invention also relates to uses of such modified multi-chain and multi-subunit proteins. For example, the invention provides methods of delivering a compound of interest to the cytoplasm of a eukaryotic cell, methods of treating a subject, and methods of generating an immune response in a subject using an inventive multi-subunit ABₙ toxin.

The invention provides a multi-chain protein that comprises at least two chains generated by proteolytic cleavage of a precursor polypeptide, wherein a compound of interest is ligated at or near each of one or more termini generated by such proteolytic cleavage. The invention provides compositions and methods for preparing such multi-chain proteins. These aspects of the invention are exemplified herein particularly with regard to bacterial exotoxins, e.g., bacterial exotoxins having an AB₅ or AB₁ structure, but the methods of the invention may be applied to other proteins that are subject to proteolytic processing. Proteins of interest may be, e.g.; receptors, channels, growth factors, hormones, or enzymes. In some embodiments, the protein of interest is a soluble protein rather than a protein that is normally membrane-bound.

The invention also provides modified AB₅ bacterial exotoxin A1 chains, and detoxified variants thereof, that have a compound of interest linked thereto. The invention also provides modified bacterial AB₅ holotoxins, in which an A1 chain of the holotoxin has a compound of interest linked thereto.

The invention provides methods to couple a compound of interest, e.g., an antigen of interest, to the A1 chain in a pre-assembled holotoxin complex. As described in further detail in the Examples, the methods have been applied to successfully ligate a variety of compounds of interest to the A1 chain of cholera toxin in a pre-assembled holotoxin complex. Importantly, the modified toxin retains the ability to enter target cells and deliver the A1 chain, with the compound of interest attached, to the cell cytoplasm.

The invention further provides pharmaceutical compositions comprising a modified AB₅ toxin protein that comprises an A1 chain having a therapeutic agent attached thereto.

The invention further provides immunogenic compositions comprising a modified AB₅ toxin protein that comprises an A1 chain having an antigen attached thereto.

The practice of the present invention will typically employ, unless otherwise indicated, conventional techniques of cell biology, cell culture, molecular biology, microbiology, recombinant nucleic acid (e.g., DNA) technology, immunology, etc., which are within the skill of the art. Such techniques are explained in the literature. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., editions as of 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Burns, R., Immunochemical Protocols (Methods in Molecular Biology) Humana Press; 3rd ed., 2005. All patents, patent applications, and other publications mentioned herein are incorporated by reference in their entirety. Standard art-accepted meanings of terms and abbreviations of terms are used herein unless otherwise indicated.

### BRIEF DESCRIPTION OF THE DRAWINGS

FIG. 1 is a schematic representation of cholera toxin.

FIG. 2 illustrates the mechanism of site-specific attachment of oligoglycine probes by sortase-mediated transpeptidation.

FIG. 3 is a diagram of the cholera toxin region in the bicistronic vector used for expression. The A (CTA=A1 chain+A2 chain) and B (CTB) subunits are represented in yellow and pink arrows, respectively. The location of the sortase recognition motif (LPETG) in the loop is highlighted in green. The secretion signal sequences that target the A and B subunit proteins to the periplasm are represented as blue arrows (lib). The Shine-Dalgarno sequences are represented as an orange box. The scale indicates base pairs.

FIGS. 4A-4D are a schematic representation of some of the cholera toxin variants tested in sortase-mediated reactions. Here only the A subunit is represented, since the B subunit structure remains native. FIG. 4d is a schematic representation of the structure of cholera toxin and of the method used to couple compounds of interest, e.g., antigenic proteins or peptides, to the catalytic portion of the toxin (i.e., A1 chain).

FIG. 5 shows an SDS-PAGE gel demonstrating purification of cholera toxin. Lane T—Periplasmic proteins released upon disruption of the outer membrane with polymixin B. Lane FT—Flow-through upon binding to Ni-NTA beads. Lane E—Eluate from the beads. Lane MQ—Pooled eluate fractions containing holotoxin, upon purification through a Mono Q column. The samples were analyzed onto a 12% SDS-PAGE under reducing conditions. The gel was stained with Coomassie blue. The molecular standards are shown in kDa. The two subunits of cholera toxin are indicated by arrows.

FIG. 6 shows analysis of cholera toxin upon digestion with trypsin. Purified cholera toxin was incubated with trypsin (Trypsin:Cholera toxin=1:1000), for 1 hr at 37° C. The samples were resolved by SDS-PAGE under reducing (+DTT) or non-reducing (−DTT) conditions. The gel was stained by Coomassie-blue. Nat—native loop (i.e., no LPETG), Mod—modified loop containing the sortase recognition motif LPETG, the HA epitope and a trypsin cleavage site. The arrows indicate the identity of the protein bands in the gel and their theoretical molecular mass. The molecular markers are indicated on the left in kDa.

FIGS. 7A-7B illustrate fluorophore attachment through sortase-catalyzed transpeptidation. A) SDS-PAGE analysis followed by Coomassie blue staining. (B) Fluorescence imaging of the gel shown in A). The position of the molecular weight standards is indicated on the left (kDa).

FIG. 8 is a schematic representation of the strategy used to prepare DTA to be used as a nucleophile in the sortase mediated transpeptidation.

FIG. 9 shows SDS-PAGE analysis of sortase-mediated transpeptidation of GGGGG-DTA onto the A1 chain of cholera toxin. Upper panel—the reaction samples were analyzed by SDS-PAGE under reducing conditions. The gel was stained with Coomassie-blue. The arrows indicate the identity of the proteins on the gel. The identity of the A1.DTA protein band was confirmed by mass-spectrometry. Lower panel—The same samples were analyzed by immunoblotting using an anti-HA antibody. The molecular standards are indicated on the left in kDa.

FIG. 10 shows results of a cytotoxicity test of the protein mixtures, derived from coupling DTA onto the A1 chain of cholera toxin, by means of sortase. Different volume reactions were added to KBM-7 cells plated on a 96-well plate. The concentration shown in the X-axis is based on the concentration of cholera toxin added from the tubes that contained this protein; same volumes were added from the mock reaction tubes. The series #1 to #6 correspond to lanes 1 to 6 from FIG. 9, as it follows: DTx—purified LFN.DTA, #1—sortase only, #2—cholera toxin only, #3—G5.DTA only, #4—sortase+G5.DTA, #5—cholera toxin+G5.DTA, #6—cholera toxin+G5.DTA+sortase. The average and the standard deviation from three independent assays are shown.

FIG. 11 shows results of an experiment in which lymph node cells from an OT-I RAG1−/− mouse were isolated, labeled with carboxyfluorescein succinimidyl ester, a fluorescent cell staining dye (CFSE) and transferred intravenously into naïve recipients. The following day, the mice were immunized in the left footpad with CTx.SIINFEKL and in the right footpad with either CTx-LPETG plus SIINFEKL or SIINFEKL alone. Two days later, popliteal lymph node cells were isolated and analyzed by flow cytometry for CFSE dilution versus CD8 expression. The extent of proliferation is reported as the number of mitotic events per progenitor cell where M/P=[ΣCi−Σ(Ci/2i)]/[Σ(Ci/2i)], where Ci denotes the number of cell counts in each gated cell division. P values were calculated using a matched pairs T test.

### DETAILED DESCRIPTION OF CERTAIN EMBODIMENTS OF THE INVENTION

### I. Definitions

An immunologic “adjuvant” is defined as any substance that acts to accelerate, prolong, or enhance antigen-specific immune responses when used in combination with a specific vaccine antigen or antigens.

“Biologically active” or “functional” when referring, e.g., to a polypeptide, means that the polypeptide displays a functionality or property that is useful as relating to some biological or biochemical process, pathway or reaction. Biological activity can refer to, for example, an ability to interact or associate with (e.g., bind to) another polypeptide or molecule (e.g., a receptor or substrate), or it can refer to an ability to physically interact with or catalyze or regulate the interaction of other proteins or molecules (e.g., enzymatic reactions). Biological activity can also refer to the ability to achieve a physical conformation characteristic of a naturally occurring structure or complex, such as the conformation of a naturally occurring multi-chain or multi-subunit protein, e.g., by undergoing appropriate folding and/or forming appropriate intramolecular or intermolecular contacts or bonds.

“Cleavage site” refers to the amino acids in a polypeptide that are joined by a peptide bond that is hydrolyzed by a protease or chemical as well as those amino acids (if any) on either side that contribute significantly to recognition and substrate specificity of the cleaving agent. According to widely used nomenclature, amino acid residues in a substrate undergoing cleavage are designated P1, P2, P3, P4, etc., in the N-terminal direction from the cleaved bond while the residues in C-terminal direction from the cleaved bond are designated P1′, P2′, P3′, P4′, etc. A cleavage site thus comprises at least the P1 and P1′ amino acids joined by the peptide bond that is cleaved. Cleavage sites for numerous cleaving agents are known in the art (see below).

An “effective amount” in the context of treating a subject is an amount sufficient to effect a beneficial or desired clinical result, e.g., the generation of an immune response, or reduced likelihood of infection, reduced severity of infection, or clinically meaningful improvement in clinical condition, e.g., an amount sufficient to palliate, ameliorate, stabilize, reverse or slow progression of the disease, or otherwise reduce pathological consequences of the disease. An immunogenic amount is an amount sufficient in the subject group being treated (either diseased or not) to elicit an immunological response, which may comprise either a humoral response, a cellular response, or both. In some embodiments an effective amount elicits production of IgA specific for an antigen of interest. An effective amount may be given in single or multiple doses.

“Engineered” is used to describe a non-naturally occurring polynucleotide or polypeptide that differs in sequence from a naturally occurring polynucleotide or polypeptide, or a cell or organism that expresses or contains such a polynucleotide or polypeptide. “Engineered” encompasses nucleic acids (e.g., DNA or RNA) that have been constructed in vitro using genetic engineering techniques or chemical synthesis, polynucleotides transcribed from such nucleic acids, and polypeptides encoded by such nucleic acids. It will be understood that an engineered polynucleotide or polypeptide may contain one or more portions derived from naturally occurring nucleic acids or proteins and/or may contain one more portions identical in sequence or having substantial sequence similarity to one or more portion(s) of one or more naturally occurring molecule(s).

A “host cell” refers to a cell that expresses an engineered or modified polynucleotide or protein. In some embodiments, a host cell is transformed to contain a vector that encodes a precursor polypeptide whereby the precursor polypeptide is produced in the cell. A host cell can be prokaryotic or eukaryotic cell, e.g., bacterial, fungal, plant, or animal (e.g., insect or mammalian). Exemplary host cells include bacterial cells (e.g., Gram-negative bacteria such as E. coli or Gram-positive bacteria such as B. subtilis or Lactococcus lactis), insect cells (e.g., Sf9), mammalian cells (e.g., CHO cells, COS cells, SP2/0 and NS/0 myeloma cells, human embryonic kidney (e.g., HEK 293) cells, baby hamster kidney (BHK) cell, human B cells, seed plant cells, and Ascomycete cells (e.g., Neurospora, Aspergillus and yeast cells; e.g., yeast of the genera Saccharomyces, Pichia, Hansenula, Schizosaccharomyces, Kluyveromyces, Yarrowia, and Candida). Exemplary yeast species include S. cerevisiae, Hansenula polymorpha, Kluyveromyces lactis, Pichia pastoris, Schizosaccharomyces pombe, and Yarrowia lipolytica.

“Identity” refers to the extent to which the sequence of two or more nucleic acids or polypeptides is the same. Percent identity may be calculated as known in the art. For example, the percent identity between a sequence of interest and a second sequence over a window of evaluation may be computed by aligning the sequences, determining the number of residues (nucleotides or amino acids) within the window of evaluation that are opposite an identical residue, allowing the introduction of gaps to maximize identity, dividing by the length of the window, and multiplying by 100. The window of evaluation may be, e.g., the length of the shorter sequence, including any gaps that were introduced to optimize the alignment (i.e., to achieve maximum percent identity), or any selected value, or if one of the polypeptides is a naturally occurring polypeptide, the length of the naturally occurring polypeptide. When computing the number of identical residues needed to achieve a particular percent identity, fractions are to be rounded to the nearest whole number. Sequence alignment can be performed using algorithms known in the art. For example, sequences can be aligned using AMPS (Barton G J: Protein Multiple Sequence Alignment and Flexible Pattern Matching. Meth Enz 183:403-428, 1990), CLUSTALW (Thompson J D, Higgins D G, Gibson T J: CLUSTAL W: improving the sensitivity of progressive multiple sequence alignment through sequence weighting, positions-specific gap penalties and weigh matrix choice. Nuc Ac Res 1994, 22:4673-4680, 1994) or GAP (GCG Version 9.1; which implements the Needleman & Wunsch, 1970 algorithm (Needleman S B, Wunsch C D: A General Method Applicable to the Search for Similarities in the Amino Acid Sequence of Two Proteins. J Mol Biol 48:443-453, 1970), the Smith-Waterman algorithm (Smith T F, Waterman M S (1981). “Identification of Common Molecular Subsequences”. Journal of Molecular Biology 147: 195-197) with default parameters, or by inspection. “Substantially identity” refers to at least 70%, 75%, 80%, 85%, 90%, 95%, 97%, 98%, 99%, or 100% identity. A “substantial portion” of a polypeptide or polynucleotide refers to at least 70%, 75%, 80%, 85%, 90%, 95%, 97%, 98%, 99% or 100% of the polypeptide or polynucleotide, starting at any position consistent with the required length. For example, a substantial portion of a 100 amino acid polypeptide could be any fragment of the polypeptide consisting of at least 70 continuous amino acids, e.g., amino acids 1-70, 2-71, 3-72 . . . 29-98, 30-99, or 31-100. It is understood that gaps may be introduced for purposes of alignment.

“Ligate” as used herein means to join or attach. A first entity is ligated to a second entity if it is structurally connected thereto.

“Modified”, as used herein with respect to a polypeptide, is often used to indicate that a compound of interest has been ligated to the polypeptide and/or that the sequence of the polypeptide is altered relative to that of a naturally occurring polypeptide. For example, a polypeptide that has been modified by transamidase-catalyzed attachment of a compound is considered “modified”.

“Multi-chain protein”, as used herein, refers to a polypeptide comprised of two or more discrete polypeptides (“chains”) that are physically associated by covalent and/or non-covalent molecular association(s) other than peptide bonds. A “multi-chain polypeptide” can contain two or more discrete polypeptides that are generated from the same precursor polypeptide molecule by proteolytic cleavage (or from different precursor polypeptide molecules that have the same sequence) or can contain two more discrete polypeptides that do not originate from a common precursor polypeptide. Thus the chains of a multi-subunit protein may be encoded by a single gene or collectively by two or more genes.

“Multi-subunit protein” refers to a multi-chain polypeptide that comprises at least two discrete polypeptide subunits that do not originate from the same precursor polypeptide (or from different precursor polypeptide molecules having the same sequence). A subunit can consist of a single polypeptide chain or can contain multiple polypeptide chains, which may be identical or different in sequence. Thus the chains of a multi-subunit protein are often collectively encoded by two or more genes.

“Polynucleotide” and “nucleic acid” are used interchangeably herein. A polynucleotide can comprise or consist of DNA, RNA, or may contain DNA and RNA. A polynucleotide can comprise standard nucleosides (i.e., the 5 nucleosides found most commonly in naturally occurring DNA or RNA) joined by phosphodiester bonds, may contain one or more non-standard nucleosides or internucleosidic linkages. In many embodiments of the invention a polynucleotide is composed of DNA

“Polypeptide” and “protein” are used interchangeably herein and can refer to molecule composed of a single polypeptide chain or multiple polypeptide chains. A “peptide” refers to a relatively short polypeptide chain, e.g., between 2 and 50 amino acids long. Amino acids in polypeptides of interest herein are often selected from among the 20 amino acids that occur most commonly in proteins found in living organisms (the “standard” amino acids). In some embodiments, a polypeptide can contain one or more naturally occurring but non-standard amino acids. In some embodiments the naturally occurring but non-standard amino acid is an amino acid that is present in some naturally occurring proteins. For example, selenocysteine and pyrrolysine are encoded by particular codons in some bacteria and are incorporated into certain proteins. Some non-standard amino acids comprise modifications such as carboxylation (e.g., of glutamate), hydroxylation (e.g., of proline), alkylation (e.g., methylation), acylation, etc., relative to a standard amino acid. In some embodiments a polypeptide contains a naturally occurring non-standard amino acid that is not found in naturally occurring proteins. Examples of nonstandard amino acids that occur naturally but in general are not found naturally in proteins include lanthionine, 2-aminoisobutyric acid, dehydroalanine, gamma-aminobutyric acid, ornithine, and citrulline. In some embodiments a polypeptide contains a non-naturally occurring (unnatural), i.e., synthetic amino acid. A vast number of unnatural amino acids having side chains not found in nature can be chemically synthesized and are available commercially from vendors such as Sigma-Aldrich. An unnatural amino acid may be a derivative of a naturally occurring amino acid, which may be a standard or non-standard amino acid. Additional examples of non-standard amino acids include naphthylalanine, norleucine, norvaline, etc. In most embodiments, amino acids in polypeptides described herein are L-amino acids. In most embodiments, amino acids in a polypeptide described herein are joined by peptide bonds.

“Precursor polypeptide”, as used herein, refers to a polypeptide that undergoes at least one proteolytic cleavage event in the process of generating a mature protein, other than removal of a signal peptide, e.g., in addition to removal of a signal peptide if one was initially present. Thus in the case of a precursor polypeptide that comprises a signal sequence, the signal sequence may first be removed and the resulting shorter precursor polypeptide subsequently undergoes a second cleavage event. For example, a polypeptide that is cleaved to generate an A1 and A2 chain of an AB₅ toxin or a polypeptide that is cleaved to generate an A chain and a B chain of an AB₁ toxin is considered a precursor polypeptide both before and after the signal sequence, if present, has been removed.

“Proteolytic processing”, “proteolytic cleavage”, or simply “cleavage” as used herein refer to breakage, e.g., hydrolysis, of a peptide bond that links amino acid residues together in a polypeptide chain.

An “individual” or “subject” is a vertebrate, e.g., a mammal or bird, e.g., a human. Non-human mammals include, but are not limited to, ovines, bovines, swine, equines, felines, canines, rodents such as mice or rats. The animal may be one of economic importance.

“Treatment” or “treating”, as used herein, encompasses clinical intervention in an attempt to alter the natural course of the individual or cell being treated, and may be performed either for prophylaxis or during the course of a disease or undesirable condition. Desirable effects include preventing occurrence or recurrence of disease, alleviation of symptoms, diminishing of any direct or indirect pathological consequences of the disease, eradicating pathogens, preventing metastasis, reducing the rate of disease progression, amelioration or palliation of the disease state, and remission or improved prognosis.

A “variant” of a particular polynucleotide or polypeptide has one or more alterations (e.g., additions, substitutions, and/or deletions) with respect to that polynucleotide or polypeptide, which polynucleotide or polypeptide may be referred to as the “original polypeptide”. A variant can be the same length as the original polynucleotide or polypeptide or may be shorter or longer. The sequence of a variant is typically at least 70% identical to the sequence of the original polynucleotide or polypeptide over a region at least 50% as long as the naturally occurring polynucleotide or polypeptide. In certain embodiments of the invention a variant is at least 50%, at least 60%, at least 70%, at least 80%, at least 90%, at least 95%, at least 97%, at least 98%, or at least 99% identical to the original polynucleotide or polypeptide over a substantial portion of the length of the original polypeptide, e.g., a region at least 50%, at least 60%, at least 70%, at least 80%, at least 90%, at least 95%, at least 97%, or at least 99%, or 100% as long as the original polynucleotide or polypeptide. In some embodiments a variant lacks 1, 2, 3, 4, or 5 amino acids present at the N- or C-terminus of the original polypeptide. Variants of naturally occurring polynucleotides and polypeptides are of particular interest herein. In some embodiments a variant has an actual or predicted 3D structure that is highly similar to, e.g., essentially superimposable on, that of the original protein with only minor differences, if any. Often a variant retains intrachain and/or interchain disulfide bonds that are present in the original polypeptide. In some embodiments most antibodies that bind to the original protein will also bind to a variant. If an activity (e.g., a biochemical or biological activity) of an original polypeptide is also possessed by a variant polypeptide, the variant is said to be biologically active with respect to that activity. A biologically active variant may be biologically active with respect to one, more than one, or all known activities of the original polypeptide. An active variant may have an activity that is at least 10%, at least 25%, at least 50%, at least 60%, at least 70%, at least 80%, or at least 90%, at least 100% of the activity of the original polypeptide, on a per molecule basis. An active variant may have increased activity relative to the original polypeptide. For example, the activity of the variant may exceed that of the original polypeptide by a factor of 1.001 to 1000. In some embodiments an activity of a variant is within a factor of 0.5 to 5 of that of the original polypeptide. An activity of a variant may be substantially reduced relative to the original polypeptide. For example, the activity may be reduced to less than 10% of the activity of the original polypeptide, e.g., 5% or less, 1% or less, 0.1% or less, 0.01% or less, etc. Stated another way, the activity may be reduced by a factor or more than 10, e.g., by a factor of 20, 30, 50, 100, 500, 1000, 10,000, etc. In some embodiments an activity is reduced to undetectable, e.g., background levels. A variant of a naturally occurring polynucleotide or polypeptide is sometimes called a “version” or “engineered version” of such polynucleotide or polypeptide herein.

A “vector”, as used herein, refers to an element capable of serving as a vehicle of genetic transfer, gene expression, or replication or integration of a foreign polynucleotide into a host cell. A vector can be, e.g., a plasmid, virus, or artificial chromosome or plasmid. In some embodiments a vector is capable of integrating into the host cell genome. In some embodiments a vector exists as an independent genetic element (e.g., episome, plasmid).

### II. Compositions and Methods for Modifying Multi-Chain Proteins

The invention relates to compositions and methods useful for ligating a compound of interest to a polypeptide that is generated by proteolytic cleavage of a precursor polypeptide. The invention also relates to modified polypeptides produced by proteolytic cleavage of a precursor polypeptide, wherein a compound of interest is ligated at or near a polypeptide terminus generated by such proteolytic cleavage. In some embodiments of the invention the modified polypeptide is a chain of a multi-chain protein that comprises two or more polypeptides generated by proteolytic cleavage of the precursor polypeptide, wherein the two or more chains remain physically associated with one another via disulfide bond(s) and/or noncovalent interactions after cleavage. At least one of the chains of the modified multi-chain polypeptide has a compound of interest ligated at or near a polypeptide terminus generated by such cleavage. In some embodiments of the invention the polypeptide is a component of a multi-subunit protein and is proteolytically cleaved after assembly of the multi-subunit protein, and a compound of interest is ligated at or near a polypeptide terminus generated by such cleavage. In some embodiments the precursor polypeptide is an engineered version of a naturally occurring precursor polypeptide. In some embodiments the naturally occurring precursor polypeptide is a precursor whose cleavage gives rise to two or more polypeptide chains of an exotoxin. In some embodiments of particular interest the exotoxin is a bacterial ABₙ exotoxin.

Pathogens have developed a variety of strategies to hijack or disable the host's cellular functions during the course of infection. The discovery of these strategies and the molecules involved has contributed significantly to advance our understanding of various cellular and physiological mechanisms. Bacterial exotoxins are among the pathogen-derived products that have been commonly used as research tools in cell biology. For example, the ability of cholera toxin and pertussis toxin to evoke elevated intracellular cyclic AMP concentration in many eukaryotic cell types has been widely exploited. In order to exert their effects on target cells, the active portion of a bacterial exotoxin must typically cross a cellular membrane to interact with their intracellular substrates. There are a variety of mechanisms by which toxins enter cells, and studying these processes is of great interest for understanding bacterial pathogenesis and for the insights it can provide into normal cellular mechanisms such as protein trafficking, among others.

Proteolytic processing plays an important role in the maturation and activation of many bacterial exotoxins, as is true for various eukaryotic proteins, e.g., enzymes of the coagulation and complement cascades, hormones such as insulin, and others, as well as a variety of virally encoded proteins. Sometimes the two (or more) individual amino acid chains resulting from proteolytic processing remain physically associated via disulfide bond(s) and/or noncovalent interactions after cleavage. In the case of bacterial exotoxins, typically one of the chains possesses a catalytic activity responsible for the protein's toxic effects while other chain(s) interact with membrane receptors at the target cell surface. For example, many bacterial exotoxins have an ABₙ structure. ABₙ toxins are comprised of A and B subunits, in which the A subunit comprises a catalytic polypeptide and associates with a B subunit comprised of one or more cell-binding polypeptides B. Toxins in which the B subunit consists of a single polypeptide chain are referred to as AB (or AB₁) toxins, while AB₅ toxins contain an A chain associated with a pentamer of B chains. AB₁ toxins and the A subunit of AB₅ toxins are synthesized as precursor polypeptides and require proteolytic cleavage to generate A and B polypeptides from the AB precursor or to cleave a precursor A polypeptide into A1 and A2 chains, respectively, in order to generate the active form (Lord, J M, et al., Curr. Topics Microbiol, Immunol., 300:149-169, 2006). Thus maturation of both AB₁ and AB₅ toxins involves proteolytic cleavage of a precursor polypeptide. In the case of AB₁ toxins, the AB polypeptide is cleaved to generate A and B chains that are linked by one or more disulfide bonds. The A chain contains the enzymatically active portion of the toxin while the B chain typically contains receptor binding and translocation domains. In the case of AB₅ toxins, the A polypeptide assembles with the pentameric B subunit, after which the A polypeptide is cleaved to generate A1 and A2 chains that are linked to one another by one or more disulfide bonds and noncovalent interactions. The A1 chain contains the enzymatically active portion of the toxin while the A2 chain serves to join the A1 chain by noncovalent interactions to the pentameric B subunit, which binds to cell surface receptors of target cells.

In order to more effectively study bacterial exotoxins and use them for various applications the inventors desired to equip these proteins with a compound of interest such as a label. However, labeling proteins that are subject to processes such as multi-subunit assembly and/or proteolytic cleavage during their maturation can be challenging. A widely used strategy to generate labeled proteins employs genetically encoded labels such as green fluorescent protein. However, this approach is inherently limited to polypeptide labels and can inhibit proper folding, subunit assembly, and/or cleavage. Likewise, other labeling approaches that involve generating a modified polypeptide prior to folding, assembly, or proteolytic processing risk disrupting these processes. The inventors sought an approach that could efficiently equip a polypeptide such as an ABₙ bacterial toxin, whose maturation involves proteolytic processing of a precursor polypeptide and that contains multiple polypeptide chains associated with one another by disulfide bonds and/or non-covalent interactions, with a compound of interest.

The invention encompasses the discovery of methods by which a transamidase can be used to efficiently ligate a compound of interest to a polypeptide whose maturation involves proteolytic processing, wherein the mature polypeptide contains at least one polypeptide chain resulting from such processing. The bacterial enzyme sortase catalyzes a transamidation reaction that has been used to derivatize proteins with many different types of modification. Target proteins are typically engineered to contain the sortase A recognition motif (LPXTG) near their C-termini. When incubated with synthetic peptides containing one or more N-terminal glycine residues and sortase A, these artificial sortase substrates undergo a transacylation reaction resulting in the exchange of residues C-terminal to the threonine residue with the synthetic oligoglycine peptide. The invention provides engineered precursor polypeptides that, following proteolytic cleavage, can serve as artificial sortase substrates to which a compound of interest can be efficiently ligated by a sortase. An engineered precursor polypeptide of the invention comprises a transamidase recognition sequence in close proximity to a protease cleavage site in the precursor polypeptide. Such positioning allows the sortase recognition sequence to be utilized with high efficiency by sortase after the polypeptide precursor is cleaved, thereby ligating a compound of interest at or near a polypeptide terminus generated by such cleavage. Importantly, according to certain embodiments of the invention, ligation takes place after the protein has folded, assembled, and been proteolytically cleaved, thereby avoiding potential interference with these processes, which are essential to generate a functional protein. Transamidase-mediated ligation of a compound of interest to a substrate is sometimes referred to herein as “sortagging”.

In some embodiments, an engineered precursor polypeptide is a variant of a naturally occurring precursor polypeptide, wherein a protease cleavage site present in the naturally occurring precursor polypeptide has been modified and wherein a different protease cleavage site has been introduced near or at the position at which the native protease cleavage site had been located. These aspects of the invention are exemplified particularly with regard to exotoxins having an ABₙ structure, but the methods may be applied to other proteins that undergo proteolytic processing.

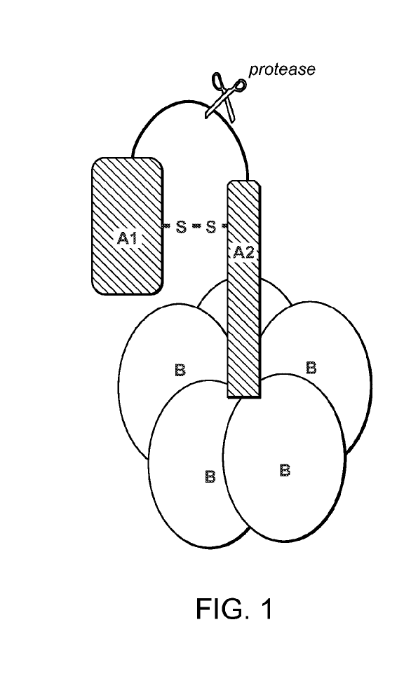

Cholera toxin (abbreviated herein as CT or CTx) is of particular interest. Cholera toxin is a major virulence factor secreted by the bacterium Vibrio cholerae and is one of the pathogen-derived products that have been commonly used as a research tool in cell biology. Upon intoxication, cholera toxin acts on the mucosal epithelium lining of the small intestine, causing the characteristic diarrhea of the disease cholera (Kaper J B, et al., Cholera, Clin Microbiol Rev., 8(1):48-86, 1995; Sánchez, J. & Holmgren, J., Cholera toxin structure, gene regulation and pathophysiological and immunological aspects, Cell. Mol. Life Sci. 65:1347-1360, 2008). Structurally, cholera toxin is an oligomeric protein displaying an AB₅ holotoxin assembly type (FIG. 1a). Cholera toxin A polypeptide is synthesized as a 258 amino acid precursor protein that includes an 18 amino acid signal sequence (Mekalanos, J. J., et al., Nature, 306, 551-557, 1983). The sequence of an exemplary CT A precursor polypeptide (accession number: P01555) is as follows:

(SEQ ID NO: 1)
MVKIIFVFFIFLSSFSYANDDKLYRADSRPPDEIKQSGGLMPRGQSEYF
DRGTQMNINLYDHARGTQTGFVRHDDGYVSTSISLRSAHLVGQTILSGH
STYYTYVIATAPNMFNVNDVLGAYSPHPDEQEVSALGGIPYSQIYGWYR
VHFGVLDEQLHRNRGYRDRYYSNLDIAPAADGYGLAGFPFEHRAWREEP
WIHHAPPGCGNAPRSSMSNTCDEKTQSLGVKFLDEYQSKVKRQIFSGYQ
SDIDTHNRIKDEL

Removal of the 18 amino acid signal sequence (underlined in SEQ ID NO: 1) results in the 240 amino acid precursor polypeptide whose sequence is shown below:

NDDKLYRADSRPPDEIKQSGGLMPRGQSEYFDRGTQMNINLYDHARGTQ TGFVRHDDGYVSTSISLRSAHLVGQTILSGHSTYYIYVIATAPNMFNVNDVLGAYSPH PDEQEVSALGGIPYSQIYGWYRVHFGVLDEQLHRNRGYRDRYYSNLDIAPAADGYG LAGFPPEHRAWREEPWIHHAPPGCGNAPRSSMSNTCDEKTQSLGVKFLDEYQSKVK RQIFSGYQSDIDTHNRIKDEL (SEQ ID NO: 2). Amino acid numbering used herein will be based on sequences as they exist following removal of the signal sequence, e.g., SEQ ID NO: 2 in the case of CT A chain.

The sequence of the B polypeptide (accession number P01556) is shown below, with the 21 amino acid signal peptide underlined. Removal of this peptide yields the 103 amino acid B polypeptide (amino acids 22-124 of SEQ ID NO: 3)

(SEQ ID NO: 3)
MIKLKFGVFFTVLLSSAYAHGTPQNITDLCAEYHNTQIYTLNDKIFSYT
ESLAGKREMAIITFKNGAIFQVEVPGSQHIDSQKKAIERMKDTLRIAYL
TEAKVEKLCVWNNKTPHAIAAI
SMAN.

The five monomeric B subunits are arranged in a doughnut-like structure, with the C-terminus of the A-subunit protruding through the central pore. This tethers the A and B subunits together. The A subunit extends well above the plane formed by the B-subunit exhibiting a protease-sensitive loop. Cleavage in this region takes place in the extracellular space and is accomplished by a hemagglutinin protease that is also secreted by Vibrio cholerae. Proteolysis yields two distinct polypeptides (the A1 and A2 chains) that remain bound by a disulfide bridge (between Cys187 and Cys199, which are underlined in SEQ ID NO: 2). Cleavage of the A polypeptide to generate the A1 and A2 chains occurs preferentially between Ser194 and Met195, and in addition between Ser193 and Ser194 (Naka A et al Toxicon (1998) 36:1001-1005). (However, data indicate that serine endoproteases, which are abundant both in bacteria and mammalian cells, are able to efficiently cleave the protease sensitive loop of cholera toxin on the C-terminal side of Arg192 (shown in bold in SEQ ID NO: 2)). The A1 chain (amino acids 1-192 of SEQ ID NO: 2) contains the catalytic active site of the toxin. The sequence of the mature A2 chain (accession number CAA53975) is shown below:

(SEQ ID NO: 4)
MSNTCDEKTQSLGVKFLDEYQSKVKRQYFSGYQSDIDTHNRIKDEL

The B-subunit pentamer works as the carrier of the toxin. It displays a very strong affinity for a membrane glycolipid receptor that is present at the cell surface, the monosialioganglioside GM1. Upon binding to this lipid the holotoxin is internalized by endocytosis into the endosomal/lysosomal system and reaches the ER by retrograde transport. In this compartment, the disulfide bridge that holds A1 and A2 chains together is reduced by the ER-resident protein disulfide isomerase (PDI), leading to the separation of the A1 chain from the rest of the complex (i.e., A2 chain and B-subunits). The exact steps that follow are still not completely understood, but it is hypothesized that once separated from the complex the A1 chain gets partially unfolded. This triggers the ER quality control system, which disposes of this presumed misfolded protein into the cytosol. Here, the A1 chain re-acquires the proper folding, escaping degradation by the proteasome, becoming active. The toxicity of the A1 chain derives from its ADP-rybosylation activity on the heterotrimeric GTP-binding protein Gsα, which triggers a signaling cascade resulting in the opening of the chloride channels located in the plasma membrane. Constitutive activation of this protein leads to continuous stimulation of adenyl cyclase with a concomitant increase in the intracellular levels of cAMP. This results in the opening of the chloride channels in the plasma membrane leading to an increase in the secretion of chloride to the extracellular space, which is accompanied by the osmotic movement of a large quantity of water.

A. Engineered Precursor Polypeptides

The invention provides engineered precursor polypeptides that can be proteolytically cleaved to yield a polypeptide chain to which a compound of interest can be ligated with high efficiency by a transamidase. The invention further provides multi-subunit proteins wherein at least one subunit comprises an engineered precursor polypeptide, wherein the engineered precursor polypeptide can be proteolytically cleaved to yield a polypeptide chain to which a compound of interest can be ligated with high efficiency by a transamidase. The invention further provides multi-chain and multi-subunit proteins that comprise an engineered polypeptide chain to which a compound of interest can be ligated with high efficiency by a transamidase. In some embodiments the engineered precursor polypeptides, multi-chain and multi-subunit proteins are variants of naturally occurring proteins. Variants of protein toxins, e.g., toxins having an ABₙ structure, are of particular interest.

In one aspect, the invention provides an engineered precursor polypeptide that comprises a polypeptide of formula

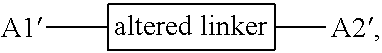

wherein the engineered precursor polypeptide is a variant of a naturally occurring precursor polypeptide of formula

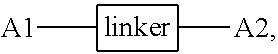

where A1 and A2 represent polypeptide domains of the naturally occurring precursor polypeptide, [symbol] comprises a peptide bond that is cleaved by a protease during maturation of the naturally occurring precursor polypeptide and is located within a first cleavage site, A1′ comprises a polypeptide whose sequence is substantially identical to the sequence of a substantial portion of A1, A2′ comprises a polypeptide whose sequence is substantially identical to the sequence of a substantial portion of A2, and [symbol] comprises a transamidase recognition sequence and a second cleavage site. In some embodiments of the invention A1′ comprises or consists of a polypeptide at least 90% identical to a substantial portion of A1, and A2′ comprises or consists of a polypeptide at least 90% identical to a substantial portion of A2. In some embodiments, A1′ comprises or consists of a polypeptide at least 90% identical to A1 over 90% of A1. In some embodiments the sequence of A1 differs from that of A1′ at 1, 2, 3, 4, 5, 6, 7, 8, 9, or 10 positions when the two sequences are optimally aligned. In some embodiments, A2′ comprises or consists of a polypeptide at least 90% identical to A2 over 90% of A2. In some embodiments the sequence of A2 differs from that of A2′ at 1, 2, 3, 4, or 5 positions when the two sequences are optimally aligned. In some embodiments A2′ is identical to A2.

Referring to the structure of a precursor polypeptide of an A subunit of an AB₅ toxin, A1 and A2 in

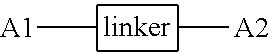

represent portions of the precursor polypeptide that give rise to the A1 and A2 chains following cleavage. Thus in some embodiments of the invention A1′ is substantially identical to an A1 chain of an AB₅ toxin over a substantial portion of the A1 chain, and A2′ is substantially identical to an A2 chain of an AB₅ toxin over a substantial portion of the A2 chain. For example, in some embodiments A1′ comprises or consists of a polypeptide that is at least 90% identical to an A1 chain of an AB₅ toxin, e.g., the A1 chain of cholera toxin. As noted above, a mature AB₅ toxin contains a disulfide bond that joins the portions that, following cleavage, constitute the A1 and A2 chains. For example, CT contains a disulfide bond between Cys187 (in the A1 portion of the A polypeptide) and Cys199 (in the A2 portion of the A polypeptide). In some embodiments of the invention A1′ is substantially identical to a portion of an A1 chain of an AB₅ toxin that lies N-terminal to the cysteine that participates in the disulfide bond (e.g., Cys187) over a substantial portion of such portion of the A1 chain, and A2′ is substantially identical to a portion of an A2 chain of an AB₅ toxin that lies C-terminal to the cysteine that participates in the disulfide bond (Cys199) over a substantial portion of such portion of the A2 chain. Thus in some embodiments

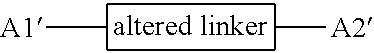

is an engineered variant of an A polypeptide of an AB₅ toxin in which a transamidase recognition sequence is inserted into the loop formed by the disulfide bond. In some embodiments the transamidase recognition sequence is positioned between the cysteine that participates in the disulfide bond and a naturally occurring protease cleavage site in the loop region. For example, in some embodiments, the transamidase recognition sequence is inserted within the sequence CGNAPRSSMSNTC in the A chain polypeptide (SEQ ID NO: 2). For example, the transamidase recognition sequence may be inserted between Cys187 and Pro191. Optionally, some of the sequence between Arg192 and Thr198, inclusive, is deleted. Optionally Pro191 and/or Arg192 is deleted. In some embodiments a protease cleavage site is inserted between the C-terminal amino acid of the transamidase recognition sequence and Cys199. In some embodiments the length of the region between the cysteines that form a disulfide bond is no more than 15, 20, 25, or 30 amino acids. Thus the invention encompasses variants of an AB₅ toxin A subunit precursor polypeptide that are substantially identical to a naturally occurring A chain precursor polypeptide (either comprising a signal sequence, or not comprising a signal sequence), wherein a transamidase recognition sequence is located between the cysteines that correspond to Cys187 and Cys199 of the naturally occurring polypeptide.

In some embodiments, the variant is substantially identical to SEQ ID NO: 2 and has a transamidase recognition sequence located between the cysteines that correspond to Cys187 and Cys199 of SEQ ID NO: 2. For example, in some embodiments A1′ is substantially identical, e.g., at least 90% or at least 95% identical, to amino acids 1-187 of SEQ ID NO: 2, and A2′ is substantially identical, e.g., at least 90% or at least 95% identical, to amino acids 199-240 of SEQ ID NO: 2. In some embodiments the variant has a transamidase recognition sequence inserted N-terminal to a protease cleavage site that occurs naturally in SEQ ID NO: 2, e.g., between Cys187 and Pro191 of SEQ ID NO: 2. Optionally the polypeptide comprises a signal sequence at the N-terminus of A1′. In some embodiments the signal sequence is from an E. coli secreted protein, e.g., E. coli LT or another AB₅ toxin produced by E. coli.

In some embodiments the variant is substantially identical to an A subunit precursor polypeptide of an LT toxin (either comprising a signal sequence, or not comprising a signal sequence) and has a transamidase recognition sequence located between the cysteines that form a disulfide bond that connects the A1 and A2 chains. In some embodiments, the variant is substantially identical to SEQ ID NO: 5 and has a transamidase recognition sequence located between the cysteines that correspond to Cys187 and Cys199 of SEQ ID NO: 5. For example, in some embodiments A1′ is substantially identical, e.g., at least 90% or at least 95% identical, to amino acids 1-187 of SEQ ID NO: 5, and A2′ is substantially identical, e.g., at least 90% or at least 95% identical, to amino acids 199-240 of SEQ ID NO: 5. In some embodiments the variant has a transamidase recognition sequence inserted between Cys187 and Pro191 of SEQ ID NO: 5. Optionally the polypeptide comprises a signal sequence at the N-terminus of A1′. In some embodiments the signal sequence is from an E. coli secreted protein, e.g., E. coli LT or another AB₅ toxin produced by E. coli.

In some embodiments A1′ comprises or consists of a polypeptide that has one or more amino acid alterations (e.g., deletions, additions, or substitutions) relative to A1 that substantially reduces the toxicity of A1′ relative to that of A1. Exemplary alterations are discussed further below. In some embodiments A1′ is identical to an A1 chain of an AB₅ toxin, e.g., the A1 chain of cholera toxin, except that A1′ has one or more such amino acid differences that substantially reduce toxicity and, in some embodiments, A1′ lacks one or more amino acids that would have been part of the cleavage site between A1 and A2 in an A subunit precursor protein. In some embodiments the amino acid differences in A1′ relative to A1 do not significantly inhibit association of A1′ with an A2 chain of an AB₅ toxin. In some embodiments the amino acid differences in A1′ relative to A1 do not significantly inhibit translocation of A1′ into the cytoplasm of a target cell when A1′ is present in an AB₅ toxin.

In some embodiments A2′ comprises or consists of a polypeptide that is at least 90% identical to an A2 chain of an AB₅ toxin, e.g., the A2 chain of cholera toxin. In some embodiments A2′ comprises or consists of a polypeptide identical to an A2 chain of an AB₅ toxin, e.g., the A2 chain of cholera toxin. In some embodiments the amino acid differences in A2′ relative to A2, if any, do not significantly inhibit association of A2′ with an A1 chain of an AB₅ toxin. In some embodiments the amino acid differences in A2′ relative to A2, if any, do not significantly inhibit assembly of A2′ with a B subunit of an AB₅ toxin. In some embodiments A2′ comprises an ER retention sequence, e.g., KDEL, at its C terminus, as in the A2 chain of cholera toxin.

In some embodiments the amino acid differences in A1′ and/or A2′ relative to A1 and/or A2, respectively, do not significantly reduce stability of an AB₅ toxin comprising A1′ and/or A2′. For example, in certain embodiments of the invention a preparation of AB₅ toxin is stable for at least 3 months, e.g., 3-6 months, or 6-12 months, or longer when stored at 4° C. in a suitable liquid medium. Methods of preparing the engineered AB₅ toxins are an aspect of the invention (see, e.g., Example 1).

Referring to the structure of a precursor polypeptide of an AB₁ toxin, A1 and A2 in

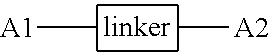

represent the portions of the precursor polypeptide that give rise to the A and B chains following cleavage. Thus in some embodiments of the invention A1′ is substantially identical to an A chain of an AB₁ toxin over a substantial portion of the A chain, and A2′ is substantially identical to a B chain of an AB₁ toxin over a substantial portion of the B chain. As noted above, a mature AB₁ toxin contains a disulfide bond that joins the A and B chains. In some embodiments of the invention A1′ is substantially identical to a portion of an A chain of an AB₁ toxin that lies N-terminal to the cysteine that participates in the disulfide bond over a substantial portion of such portion of the A chain, and A2′ is substantially identical to a portion of an B chain of an AB₅ toxin that lies C-terminal to the cysteine that participates in the disulfide bond over a substantial portion of such portion of the B chain.

[symbol] in

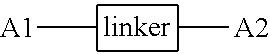

may be a single peptide bond, in which case the P1 amino acid of the cleavage site is located at the C-terminus of A1 and the P1′ amino acid of the cleavage site is located at the N-terminus of A2. The protease that naturally cleaves [symbol] is sometimes produced by an organism that naturally produces the naturally occurring precursor protein or sometimes is present in the environment into which the naturally occurring precursor protein is secreted or subsequently found (e.g., within a target cell or organism in the case of toxins). In some embodiments, [symbol] comprises a portion of the naturally occurring precursor polypeptide that is removed in the process of maturation of the protein. For example, [symbol] could have a P1′ amino acid of a cleavage site at its N-terminus and a P1 amino acid of another cleavage site at its C-terminus, or could contain two cleavage sites, such that upon cleavage at both sites [symbol] is removed from the polypeptide (although in some instances [symbol] or a portion thereof may remain attached to either A1 or A2 by a disulfide bond or noncovalent interaction).

Returning to the description of the engineered precursor polypeptide, [symbol] [symbol] in

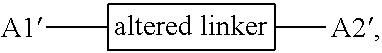

comprises a transamidase recognition sequence and a cleavage site. A variety of suitable transamidase recognition sequences and cleavage sites are described below. In some embodiments, the transamidase recognition sequence is located N-terminal with respect to the cleavage site within [symbol] . In these embodiments the N-terminal amino acid of the transamidase recognition sequence (often a glycine residue) is usually located not more than 20 amino acids away from the peptide bond that is cleaved within the cleavage site (i.e., there are usually not more than 19 amino acids between the C-terminal amino acid of the transamidase recognition sequence and the P1 amino acid of the cleavage site). In certain of these embodiments the C-terminal amino acid of the transamidase recognition sequence is located not more than 5, or in some embodiments not more than 10, or in some embodiments not more than 15 amino acids away from the peptide bond that is cleaved within the cleavage site. The polypeptide segment between the C-terminal amino acid of the transamidase recognition sequence and the N-terminal amino acid of the cleavage site is referred to as a “polypeptide spacer”. The polypeptide spacer, if present, is usually between 1 and 19 amino acids long, e.g., between 1 and 5 amino acids, between 5 and 10 amino acids, between 10 and 15 amino acids long. The polypeptide spacer can, in general, have any sequence. In some embodiments the polypeptide spacer comprises an epitope tag, e.g, an HA, FLAG, or Myc tag. Since the tag is removed during the transamidase-mediated reaction, including a tag in the polypeptide spacer allows the efficiency of the reaction to be monitored (see Example 1). In some embodiments, the polypeptide spacer does not contain a cysteine residue.

The cleavage site in [symbol] could be the same or different to the cleavage site found in the naturally occurring polypeptide. In some embodiments a protease cleavage site present in [symbol] in the naturally occurring precursor polypeptide has been modified (e.g., at least in part deleted or substituted with different amino acids), so that the engineered precursor polypeptide is not a substrate for the protease that, in nature, cleaves the naturally occurring precursor polypeptide is a physiological substrate. In some embodiments, the cleavage site in [symbol] is selected such that the engineered precursor polypeptide is not a substrate for a protease present in a host cell of interest. The host cell of interest may be any cell in which a recombinant polypeptide can be produced, e.g., a bacterial cell, yeast cell, insect cell, mammalian cell, or plant cell. For example, if the engineered precursor polypeptide is to be produced in bacteria, e.g., E. coli, the cleavage site may be one that is not cleaved by proteases (e.g., serine endoproteases) commonly found in bacteria. In some embodiments [symbol] does not contain a cysteine. In some embodiments the length of [symbol] is no more than 30, in some embodiments no more than 25, in some embodiments no more than 20, in some embodiments no more than 15, in some embodiments no more than 10, or in some embodiments no more than 5 amino acids in length. For example, in some embodiments [symbol] represents an insertion of no more than 5, 10, 15, 20, 25, or 30 amino acids between the C-terminus of the A1 and the N-terminus of the A2 portions of an A subunit precursor polypeptide of an AB₅ toxin.

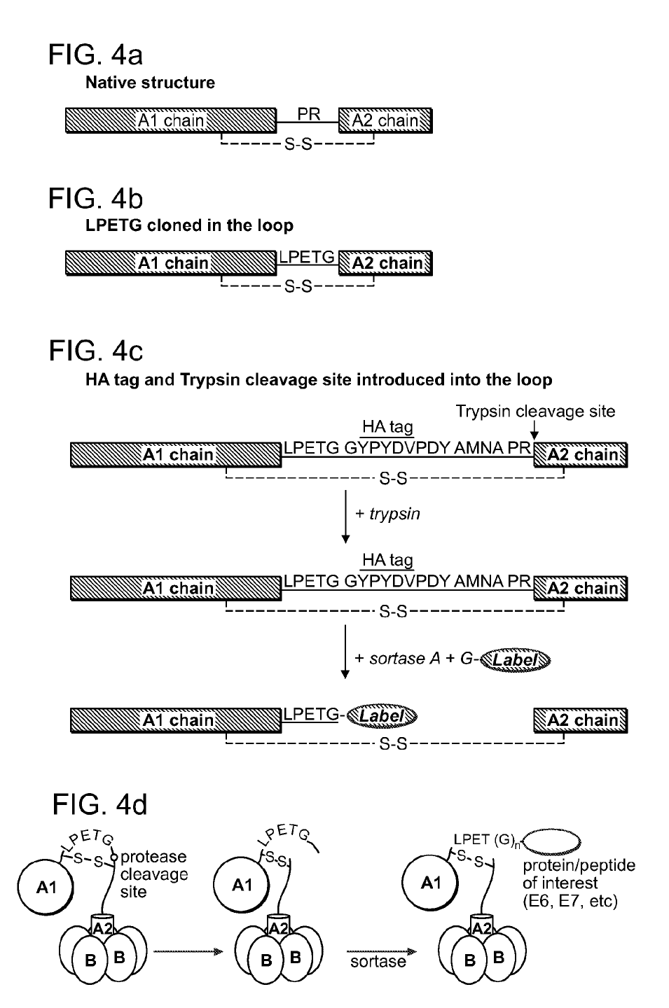

For example, a schematic representation of an engineered precursor polypeptide that is a variant of cholera toxin A chain precursor polypeptide is shown in the upper panel of FIG. 4c. In the engineered precursor polypeptide [symbol] comprises, in an N-terminal to C-terminal direction, the transamidase recognition sequence, a polypeptide spacer that comprises an HA tag, and a cleavage site for trypsin. Cleavage at the cleavage site generates an engineered variant of an A1 chain of cholera toxin having a transamidase recognition sequence close to its C-terminus. According to the inventive approach, the resulting cleaved engineered polypeptide can serve as a substrate in a reaction in which a nucleophilic compound comprising an NH₂—CH₂— moiety, e.g., a compound comprising a NH₂CH₂(C═O)— moiety. In some embodiments the compound comprises (G)ₖ-, where k is an integer from 1 to 6, is ligated to the cleaved engineered polypeptide by sortase (see lower two panels of FIG. 4c).

In other embodiments of the invention [symbol] comprises, in an N- to C-direction, a cleavage site and one or more glycine residues, e.g., (G)ₖ, wherein G represents glycine and k is between 1 and 6. In some embodiments, n is between 3 and 5. Optionally a polypeptide spacer as described above is located between the cleavage site and (G)ₖ. Cleavage at the cleavage site generates an engineered polypeptide, e.g., an engineered variant of an A2 chain of an AB₅ toxin, having one or more glycine residues at its N-terminus. According to the inventive approach, the resulting cleaved engineered polypeptide serves as a nucleophile in a sortase-mediated reaction, thereby allowing ligation of a compound of interest that comprises or is attached to a transamidase recognition sequence to the N-terminus of the cleaved engineered polypeptide. It is contemplated in some embodiments to use the inventive methods for ligation of a compound to an N-terminus disclosed in published PCT application WO 2010/087994.

The methods of the invention may be applied to generate modified engineered versions of a wide variety of naturally occurring proteins. AB₅ toxins are of particular interest. In addition to cholera toxin, Shiga toxin (ST), the Shiga-like toxins (e.g., SLT1, SLT2, SLT2c, and SLT2e, collectively referred to herein as SLTs), E. coli heat labile enterotoxins LT-I (e.g., the two variants LT-Ih from human isolates and LT-Ip from porcine isolates), LT-IIa, and LT-IIB, and pertussis toxin (PT), are examples of bacterial AB₅ toxins. With the exception of PT, the B subunit of these toxins is a homopentamer. PT exhibits the general AB₅ assembly, with an enzymatically active chain formed by cleavage of the S1 precursor polypeptide, while the receptor-binding B subunit is made up of polypeptides S2-S5, including two S4 polypeptides. LT-I, also referred to simply as “LT” is similar to CT in sequence and is of particular interest herein. In addition to using GM1 as a receptor, LT-I can also bind to GD1b and to other carbohydrate residues present in intestinal glycoproteins. ST and most SLTs utilize the glycosphingolipid globotriaosylceramide (Gb₃) as a receptor for target cell entry. The sequences of these toxins and of the nucleic acids that encode them in their organism of origin are available in the literature and in public databases. For example, some representative accession numbers from Entrez are as follows:

TABLE 1
Accession numbers of selected ABₙ toxin precursor polypeptides

| | Accession |
|---|---|
| Polypeptide | Number |
| E. coli heat labile enterotoxin type I subunit A precursor | AAA24685 |
| E. coli heat labile enterotoxin type I subunit B precursor | AAC60441 |
| E. coli heat labile enterotoxin type IIa subunit A precursor | AAA24093 |
| E. coli heat labile enterotoxin type IIa subunit B precursor | AAA24094 |
| E. coli heat-labile enterotoxin type IIb subunit A precursor | AAA53285 |
| E. coli heat-labile enterotoxin type IIb B chain precursor | AAA53286 |
| Shiga toxin subunit A precursor | YP_403025 |
| Shiga toxin subunit B precursor | YP_403026 |

An exemplary sequence of the E. coli heat labile enterotoxin subunit A precursor (pathogenic for humans) after removal of the 18 amino acid N-terminal signal sequence (MKNITFIFFILLASPLYA) is as follows: NGDKLYRADSRPPDEIKRSGGLMPRGHNEYFDRGTQMNINLYDHARGTQTGFVRYD DGYVSTSLSLRSAHLAGQSILSGYSTYYIYVIATAPNMFNVNDVLGVYSPHPYEQEVS ALGGIPYSQIYGWYRVNFGVIDERLHRNREYRDRYYRNLNIAPAEDGYRLAGFPPDH QAWREEPWIHHAPQGCGDSSRTITGDTCNEETQNLSTIYLRKYQSKVKRQIFSDYQSE VDIYNRIRNEL (SEQ ID NO: 5). Cysteines 187 and 199, which form a disulfide bond in the mature protein, are also underlined. The signal sequence MKNITFIFFILLASPLYA It will be understood that minor sequence differences may occur among different strains or isolates of any bacterial species, and the sequences listed under the accession numbers should be considered exemplary. Exemplary toxin-producing V. cholerae strains of the classical biotype are known as 569B, 41, O395. Exemplary toxin-producing V. cholerae strains of the El Tor biotype are known as 2125, 62746, and 3083. Exemplary toxin-producing E. coli strains of human origin are known as H74-114 and H10407. An toxin-producing E. coli strain of porcine origin is known as P307. See, e.g., Chapter 15 of Alouf & Popoff, supra. The invention contemplates variants whose sequence is based on the sequence of any isolate.

The 3D structures of a number of AB₅ toxins are known. These include CT (Zhang, R G, et al. The three-dimensional crystal structure of cholera toxin. J Mol Biol., 251(4):563-73, 1995), LT-I (Sixma, T K, et al., Refined structure of Escherichia coli heat-labile enterotoxin, a close relative of cholera toxin, J Mol Biol., 230(3):890-918, 1993); LT-IIb (van den Akker F, et al. Crystal structure of a new heat-labile enterotoxin, LT-IIb. Structure, 4(6):665-78, 1996), PT (Stein, P E, et al., The crystal structure of pertussis toxin. Structure 2(1), 45-57, 1995), and ST (Fraser M E, et al., Crystal structure of the holotoxin from Shigella dysenteriae at 2.5 Å Nat Struct Biol., 1(1):59-64, 1994). The structures of these proteins are highly similar (although PT contains an additional domain in two of the five monomers that make up the B subunit) and in each case reveals a proteolytic cleavage site in the A polypeptide located within a loop region that is surface-exposed in the holotoxin structure. Cleavage at this site after assembly of the A chain with the pentameric B subunit results in formation of A1 and A2 chains as described above for CT. Thus it will be evident that the methods of the invention as described and exemplified herein for CT may be readily applied to the other AB₅ toxins. In some embodiments of the invention an engineered AB₅ toxin is composed of an engineered A subunit that is a variant of an A subunit from a first naturally occurring AB₅ toxin (e.g., CT) and a B subunit that is identical to or an engineered variant of a B subunit from a second naturally occurring AB₅ toxin (e.g., LT).

The invention provides engineered variants of AB₁ toxins. Diphtheria toxin (DT) is an exemplary AB₁ toxin. It is produced by certain Corynebacterium diphtheriae strains with a 25 amino acid signal peptide and secreted as a single polypeptide chain. Upon cleavage of the signal sequence the toxin is released into the extracellular environment where serine protease attack at a site within a 14 amino acid protease-sensitive loop results in formation of two chains, A and B, corresponding to N- and C-terminal fragments respectively, of the immediate precursor polypeptide. The A and B chains remain covalently attached by an interchain disulfide bond. The receptor for DT has been shown to be the heparin-binding epidermal growth factor-like growth factor (hHB-EGF). Pseudomonas exotoxin A (ExoA), another bacterial AB₁ toxin, utilizes the low density lipoprotein receptor-related protein (LRP), also known as the α2-macroglobulin receptor to enter cells. Binding leads to endocytosis via coated pits, bringing the toxin to the compartment where it is cleaved between arginine 279 and glycine 280 into an N-terminal fragment of 28 kDa and a C-terminal fragment of 37 kDa, leaving two chains joined by the disulfide bond linking cysteines 265 and 287.

Botulinum neurotoxin (BoNT), produced by Clostridum botulinum, is another bacterial toxin of interest whose maturation involves proteolytic cleavage of a precursor polypeptide resulting in two polypeptide chains linked by a disulfide bond. BoNT is considered an AB₁ toxin herein. BoNT inhibits synaptic exocytosis in peripheral cholinergic synapses causing botulism, a disease characterized by descending flaccid paralysis. Clostridium botulinum strains express seven BoNT isoforms, each of which is synthesized as a single polypeptide chain with a molecular mass of ˜150 kDa. Structurally, the mature toxin consists of three modules: a 50 kDa light chain (LC) Zn2+-metalloprotease (which is enzymatically active and is considered an “A” polypeptide in the ABₙ nomenclature), and the 100 kDa heavy chain (HC) which encompasses the N-terminal ˜50 kDa translocation domain (TD), and the C-terminal ˜50 kDa receptor-binding domain (RBD) and is considered a “B” polypeptide in the ABₙ nomenclature).

Other bacterial AB₁ toxins of note include tetanus neurotoxin, produced by C. tetani, and the large clostridial toxins known as Toxin A and Toxin B, produced by C. difficile.

ABₙ toxins are found not only in bacteria but also, for example, in certain fungi and plants. The AB₁ toxin family includes certain type II ribosome inactivating plant toxins such as ricin, abrin, cinnanomin, viscumin, ebulin, and nigrin b (Hartley, M R & Lord, J M, Cytotoxic ribosome-inactivating lectins from plants, Biochim Biophys Acta, 1701(1-2):1-14, 2004; Xu H, et al., Cinnamomin—a versatile type II ribosome-inactivating protein. Acta Biochim Biophys Sin (Shanghai) 36(3):169-76). Ricin, for example, is produced in the castor oil plant as a precursor (proricin) in which a short linker region separates the disulfide-bonded A and B chains. The linker targets the transport of proricin to vacuoles where proteolytic activation occurs. Cleavage and reduction causes dissociation of the two subunits, and the active chain enters the cytosol where it cleaves an adenine residue in the large rRNA, thereby inactivating it and inhibiting protein synthesis with lethal effect.

Certain fungi (so-called “killer” strains) secrete toxins (“killer” toxins) that are lethal to sensitive strains of different species and genera. The S. cerevesiae K1, K2, and K28 toxins are exemplary yeast ABₙ toxins. These toxins are synthesized as precursor proteins that are posttranslationally imported into the ER lumen where signal peptidase cleavage removes the toxin's N-terminal secretion signal. In a late Golgi compartment the Kex2p endoprotease cleaves the pro-region, removes the intramolecular γ-sequence, resulting in a mature multi-chain protein in which the α and β subunits are linked by a disulfide bond resulting in an AB₁ structure. The salt-mediated killer toxin (SMKT) of the yeast Pichia farinosa is also composed of A and B (α and β) subunits generated from a precursor polypeptide, which remain associated by noncovalent interactions in the mature toxin (Suzuki, C., “Acidophilic structure and killing mechanism of the Pichia farinosa killer toxin SMKT” in Schmitt M J and Schaffrath, R, supra).

Further information regarding the toxins discussed above and many others may be found in the following references: Alouf, J E & Popoff, M R, (eds.) The Comprehensive Sourcebook of Bacterial Protein Toxins, Third Edition, Academic Press, 2006; Schmitt, M J & Schaffrath, R (eds.) Microbial Protein Toxins, Topics in Current Genetics 11, Berlin, N.Y.: Springer-Verlag, 2005; Proft, T. (ed.) Microbial toxins: molecular and cellular biology, Norfolk, England: BIOS Scientific, c2005.

In some embodiments of the invention an engineered variant of a naturally occurring ABₙ toxin has an alteration that substantially reduces its toxicity relative to that of a naturally occurring ABₙ toxin. Such alterations may be desirable to avoid cell damage or cytotoxicity if the engineered version is contacted with cells in vitro or administered to a subject. In some embodiments an alteration is a deletion. In some embodiments an alteration is a substitution. In some embodiments a substitution is a non-conservative substitution while in other embodiments a substitution is a conservative substitution. Conservative amino acid substitutions may be made on the basis of similarity in polarity, charge, solubility hydrophobicity, hydrophilicity, and/or the amphipathic nature of the residues involved. For example, non-polar (hydrophobic) amino acids include alanine, leucine, isoleucine, valine, proline, tryptophan, and methionine; polar/neutral amino acids include glycine, serine, threonine, cysteine, tyrosine, asparagine, and glutarmine; positively charged (basic) amino acids include arginine, lysine, and histidine; and negatively charged (acidic) amino acids include aspartic acid and glutamic acid. In some embodiments the alteration is in the A polypeptide, e.g., within the A1 chain of an AB₅ toxin). For example, deletion or substitution of catalytic residues will typically greatly reduce or eliminate toxicity. In some embodiments, an alteration does not substantially inhibit assembly of the A chain with the B subunit. In some embodiments, an alteration does not substantially inhibit binding of the toxin to its receptor on target cells and does not substantially inhibit internalization of the toxin. In some embodiments the alteration does not substantially inhibit the ability of the enzymatically active chain to enter the cytoplasm of a target cell.

A variety of alterations that substantially reduce the enzymatic activity and/or cytotoxic effect of an ABₙ toxin are of use. The following examples, in which amino acid positions refer to the wild type sequence (e.g., SEQ ID NO: 2 in the case of CT) are non-limiting. In some embodiments, a CT variant has a change of E at position 110, e.g., to D, a change of E at position 112, e.g., to D, or both. In some embodiments a CT variant has a change of E at position 110 to K. In some embodiments a CT variant has a deletion of the amino acids at positions 110, 111, and/or 112, e.g., a deletion of amino acids 110-112. In some embodiments a CT variant has a change of E at position 29, e.g., to H. In some embodiments a CT variant has a change of S at position 61, e.g., to F. In some embodiments a CT variant has an amino acid substitution at amino acid position 16, 68, and/or 72 (e.g., a substitution at positions 16 and 72). For example, I at position 16 in the A subunit is substituted with A and/or V at position 72 is substituted with a Y. In some embodiments a CT variant has a serine substituted at position 109. In some embodiments a CT variant has a combination of two or more of the foregoing alterations. In some embodiments a CT variant has an addition of one or more amino acids at the N-terminus relative to wild type CT, e.g., addition of 6 or 16 amino acids at position 1 or an alteration at the C-terminus of the A chain, e.g., an alteration of KDEL to KDEV or KDGL.

In some embodiments an LT variant has a change of A at position 72 to R. In some embodiments an LT variant has a change of R at position 192 to G. In some embodiments an LT variant has a change of S at position 63 to Y. In some embodiments an LT variant has a deletion of amino acids 110, 111, and/or 112, e.g., a deletion of amino acids 110-112. In some embodiments an LG variant has a combination of two or more of the foregoing alterations.

In some embodiments an engineered variant of an AB₅ toxin has an alteration in a B polypeptide relative to a wild type B polypeptide.

In some embodiments a variant of DT A chain has a deletion of Glu148 or a substitution of Glu148, e.g., replacement of Glu148 by Ser (see U.S. Pat. No. 7,115,725). In some embodiments additional residues are deleted or substituted, e.g., some or all of the amino acids between Glu142 and Glu147, inclusive. Other positions that may be altered are, e.g., His21, Glu22, Lys39, Gly52, Gly79, Gly128, Ala158, Glu162.

B. Transamidase Enzymes and Transamidase Recognition Sequences

As discussed above, methods of ligation described herein are catalyzed by a transamidase, and engineered precursor polypeptides of the invention comprise a transamidase recognition sequence. Transamidases can form a peptide linkage (i.e., amide linkage) between an acyl donor compound and a nucleophilic acyl acceptor containing a NH2-CH2-moiety. In certain embodiments of the invention the transamidase is a sortase. Sortases have been isolated from a variety of different Gram-positive bacteria in which they function to cleave and translocate proteins to proteoglycan moieties in intact cell walls. Gram-positive bacteria include members of the following genera: Actinomyces, Bacillus, Bifidobacterium, Cellulomonas, Clostridium, Corynebacterium, Micrococcus, Mycobacterium, Nocardia, Staphylococcus, Streptococcus, and Streptomyces.

Sortases have been classified into 4 classes, designated A, B, C, and D, based on sequence alignment and phylogenetic analysis of 61 sortases from Gram positive bacterial genomes (Dramsi S, et al., Sorting sortases: a nomenclature proposal for the various sortases of Gram-positive bacteria. Res Microbiol. 156(3):289-97, 2005). These classes correspond to the following subfamilies, into which sortases have also been classified by Comfort and Clubb (Comfort D & Clubb R T. A comparative genome analysis identifies distinct sorting pathways in gram-positive bacteria. Infect Immun., 72(5):2710-22, 2004): Class A (Subfamily 1), Class B (Subfamily 2), Class C (Subfamily 3), Class D (Subfamilies 4 and 5). Sequences of many sortases and of the naturally occurring nucleic acids that encode them are found in publicly available databases such as those of the National Center for Biotechnology Information (NCBI) available at Entrez (http://www.ncbi.nlm.nih.gov/Entrez), e.g., GenBank. The sequences of sortase proteins having the accession numbers provided herein are hereby incorporated by reference. Minor sequence differences may occur among different strains or isolates of any bacterial species, and the sequences listed under the accession numbers should be considered exemplary. For example, a S. aureus sortase A subsp. aureus N315 (accession number NP_{—}375640) differs slightly from that under accession number AAD48437.

Class A sortases, e.g., S. aureus sortase A, are of particular interest. The prototypical class A sortase, S. aureus sortase A, has been purified and characterized (Ton-that, H., et al., Purification and characterization of sortase, the transpeptidase that cleaves surface proteins of Staphylococcus aureus at the LPXTG motif, PNAS, 96(22):12424-12429, 1999), and the gene that encodes it has been cloned and sequenced (Mazmanian, S., et al., Staphylococcus aureus Sortase, an Enzyme that Anchors Surface Proteins to the Cell Wall, Science, 285, no. 5428, pp. 760-763, 1999. The gene has been assigned accession number AF162687. The protein sequence has accession number AAD48437.1 and is as follows: MKKWTNRLMTIAGVVLILVAAYLFAKPHIDNYLHDKDKDEKIEQYDKNVKEQASK DKKQQAKPQIPKDKSKVAGYIEIPDADIKEPVYPGPATPEQLNRGVSFAEENESLDDQ NISIAGHTFIDRPNYQFTNLKAAKKGSMVYFKVGNETRKYKMTSIRDVKPTDVGVLD EQKGKDKQLTLITCDDYNEKTGVWEKRKIFVATEVK. Sequences of class A sortases from a variety of other bacterial species are available under the following GenBank accession numbers: S. pyogenes (Spyog) SrtA, AAK34025; S. gordonii (Sgord) SrtA, AAG41778; L. lactis (Llact) hypO, AAK05211; S. aureus (Saure) SrtA, AAD48437; and A. naeslundii (Anaes) fimbria-associated protein (fimassoc), AAC13546; Staphylococcus aureus subsp. aureus MSSA476, CAG44229.

Class B sortases have been found, e.g., among species in the Streptococcus, Bacillus, Staphylococcus, Clostridia and Listeria genera, among others. Sequences of several class B sortases are available at GenBank accession numbers as follows: S. pyogenes, NP_{—}268518; B. anthracis, NP_{—}846988; C. perfringens, NP_{—}561429; E. faecalis, AAQ16264; Staphylococcus aureus subsp. aureus MRSA252, CAG40110; L. monocytogenes, CAD00259. Class C sortases have been found, e.g., among species in the Streptococcus, Enterococci, Bacillus, and Clostridia genera. Sequences of several class C sortases are available under the following accession numbers: S. pyogenes, AAL11468; C. diphtheriae, NP_{—}940532.1; Streptococcus suis, BAB83966. Class D sortases have been found, e.g., among species in the Streptomyces, Corynebacterium, Clostridium, Bacillus genera. Sequences of several class D sortases are available under the following accession numbers: Streptomyces coelicolor, NP_{—}628037; B. subtilis, CAB12748, C. tetani, NP_{—}781831.

A sortase of use in the invention can be naturally produced (i.e., produced by the bacterium that naturally expresses it) or can be produced by expressing a gene encoding the sortase in a suitable host using standard genetic engineering techniques for expression of recombinant proteins. The host can be, for example, bacteria, fungal, plant, insect, or mammalian cells. Typically the cells are maintained in cell culture. In other embodiments, a sortase is produced by a transgenic plant or animal. The sortase polypeptide can be produced and purified using standard techniques known to those skilled in the arts of molecular biology, biochemistry, and protein purification. See, e.g., Ton-that, H., supra. Any nucleotide sequence that encodes a sortase may be used for purposes of expressing a sortase. The nucleotide sequence may, if desired, be optimized according to codon usage in the organism in which the sortase is expressed. In some embodiments a tag such as an HA tag or 6×His tag is added to the sortase sequence to allow convenient purification. In addition to naturally-occurring sortase proteins, the skilled artisan will appreciate that proteins that have alterations in the amino acid sequence relative to the sequence of a naturally occurring sortase can be used, provided that the variant of sortase retains functional ability of the naturally occurring protein to mediate the transamidation reaction. Suitable alterations include substitution or deletion of amino acid residues not required for activity as well as conservative amino acid changes (e.g., replacing an amino acid residue with an amino acid residue having a similar side chain). It will also be appreciated that directed changes can be made, resulting in a sortase that recognizes a different recognition motif relative to a naturally occurring counterpart. Considerable information is available to guide in making such modifications and in avoiding modifications at residues important for activity. For example, a crystal structure of S. pyogenes sortase A is available (Banfield, M. J. et. al. Crystal structure of S. pyogenes sortase A: Implications for sortase mechanism J. Biol. Chem. Epub ahead of print, 2009. See also Zong Y, et al., Crystal structures of Staphylococcus aureus sortase A and its substrate complex. J Biol Chem. 279(30):31383-9, 2004, and Zong Y, et al., The structure of sortase B, a cysteine transpeptidase that tethers surface protein to the Staphylococcus aureus cell wall. Structure. 12(1):105-12, 2004; Zhang R, et al. Structures of sortase B from Staphylococcus aureus and Bacillus anthracis reveal catalytic amino acid triad in the active site. Structure, 12(7):1147-56, 2004)

An engineered precursor polypeptide of the invention comprises a transamidase recognition sequence. In some embodiments of the invention the transamidase recognition sequence is a sequence recognized and cleaved by a class A sortase. For example, the sequence may comprise X¹X²X³X⁴X⁵, where X¹ is leucine, isolucine, valine or methionine; X² is proline or glycine; X³ is any amino acid; X⁴ is threonine, serine or alanine; and X⁵ is glycine or alanine. In some embodiments the sequence comprises LPXTG, e.g., LPKTG, LPATG, LPNTG, LPETG. In some embodiments the motif comprises an ‘A’ rather than a ‘T’ at position 4, e.g., LPXAG, e.g., LPNAG or an ‘A’ rather than a ‘G’ at position 5, e.g., LPXTA, e.g., LPNTA or a ‘G’ rather than ‘P’ at position 2, e.g., LGXTG, e.g., LGATG or an ‘I’ rather than ‘L’ at position 1, e.g., IPXTG, e.g., IPNTG or IPETG (where X in the foregoing sequences is any amino acid).

In some embodiments of the invention the transamidase recognition sequence is a sequence recognized and cleaved by a class B sortase. Motifs recognized by class B sortases often fall within the consensus sequences NPXTX (where X represents any amino acid), e.g., NP[Q/K]-[T/s]-[N/G/s], such as NPQTN or NPKTG. For example, sortase B of S. aureus or B. anthracis cleaves the NPQTN or NPKTG motif (see, e.g., Marraffini, L. and Schneewind, O., J. Bact., 189(17), p. 6425-6436, 2007). Other recognition motifs found in putative substrates of class B sortases are NSKTA, NPQTG, NAKTN, and NPQSS. For example, SrtB from L. monocytogenes recognizes certain motifs lacking P at position 2 and/or lacking Q or K at position 3, such as NAKTN and NPQSS (Mariscotti J F, García-Del Portillo F, Pucciarelli M G. The listeria monocytogenes sortase-B recognizes varied amino acids at position two of the sorting motif. J Biol Chem. 2009 Jan. 7. [Epub ahead of print])

In some embodiments of the invention the transamidase recognition sequence is a sequence recognized and cleaved by a class C sortase. Class C sortases may utilize LPXTG as a recognition motif. In some embodiments of the invention the transamidase recognition sequence is a sequence recognized and cleaved by a class D sortase. Sortases in this class are predicted to recognize motifs with a consensus sequence NA-[E/A/S/H]-TG (Comfort D, supra). LPXTA or LAXTG may serve as a recognition sequence for class D sortases, e.g., of subfamilies 4 and 5, respectively). For example, a B. anthracis class D sortase, has been shown to specifically cleave the LPNTA motif (Marrafini, supra). A sortase that recognizes QVPTGV motif has been described (Barnett, T C and Scott, J R, Differential Recognition of Surface Proteins in Streptococcus pyogenes by Two Sortase Gene Homologs. J. Bact., Vol. 184, No. 8, p. 2181-2191, 2002).

The invention contemplates use of sortase proteins found in any Gram positive organism, such as those mentioned herein and/or in the references and/or databases cited herein. The invention also contemplates use of sortase proteins found in gram negative bacteria, e.g., Colwellia psychrerythraea, Microbulbifer degradans, Bradyrhizobium japonicum, Shewanella oneidensis, and Shewanella putrefaciens. They recognize sequence motifs LP[Q/K]T[A/S]T. In keeping with the variation tolerated at position 3 in sortases from Gram positive organisms, a sequence motif LPXT[A/S], e.g., LPXTA or LPXTS may be used.

The invention contemplates use of sortase recognition motifs from any of the experimentally verified or putative sortase substrates listed at http://bamics3.cmbi.kun.nl/jos/sortase_substrates/help.html, the contents of which are incorporated herein by reference, and/or in any of the above-mentioned references. In some embodiments the sortase recognition motif is selected from: LPKTG, LPITG, LPDTA, SPKTG, LAETG, LAATG, LAHTG, LASTG, LAETG, LPLTG, LSRTG, LPETG, VPDTG, IPQTG, YPRRG, LPMTG, LPLTG, LAFTG, LPQTS. In some embodiments, a recognition sequence further comprises one or more additional amino acids, e.g., on the N terminal side. For example, one or more amino acids (e.g., up to 5 amino acids) having the identity of amino acids found immediately N-terminal to, or C-terminal to, a 5 amino acid recognition sequence in a naturally occurring sortase substrate may be incorporated. Such additional amino acids may provide context that improves the efficiency of utilization of the recognition sequence by sortase. In some embodiments of the invention the transamidase recognition sequence is followed by a G residue. Thus the invention contemplates altering a portion of an A chain precursor polypeptide of an AB₅ toxin to include a transamidase recognition sequence followed by a G residue, e.g., LPXTGG. For example, in some embodiments LPETGG is used.

The invention comprises embodiments in which ‘X’ in a sortase recognition sequence is any amino acid. In many embodiments, X is selected from the 20 standard amino acids found most commonly in proteins found in living organisms. In certain embodiments in which the engineered precursor protein is produced in a host cell, X is an amino acid that can be incorporated into a polypeptide chain by the translation machinery of the host cell. In certain embodiments in which a synthetic nucleophile In some embodiments, e.g., if the recognition sequence is LPXTG, X is D, E, A, N, Q, K, or R. In some embodiments, X is selected from among those amino acids that occur naturally at position 3 in a naturally occurring sortase substrate. For example, in some embodiments a class A sortase is used, and X in an LPXTG sequence is selected from K, E, N, Q, A In some embodiments a class C sortase is used, and X in an LPXTG sequence is selected from K, S, E, L, A, N.

C. Cleaving Agents and Cleavage Sites

Naturally occurring precursor proteins contain one or more sites that are recognized and cleaved by a protease. In the case of ABₙ toxins, the protease may be endogenous to the organism that produces the toxin or may be found in the target organism. As discussed above, in some embodiments of the invention a protease cleavage site that is cleaved in nature in a naturally occurring precursor polypeptide is deleted, altered, or moved so that the engineered version is no longer a substrate for the protease that cleaves it in nature. In some embodiments of the invention a protease cleavage site that would be cleaved by a protease present in a particular host cell in which it is desired to express the engineered polypeptide is deleted, altered, or moved so that the engineered version is no longer a substrate for such a protease. In some embodiments of the invention an engineered precursor polypeptide comprises a protease cleavage site that is not found in the naturally occurring version of the precursor polypeptide or is found in a different context (i.e., has different amino acids on either side). The engineered protease cleavage site is positioned sufficiently close to the transamidase recognition sequence so that cleavage at the engineered protease cleavage site generates a free C-terminus located within 20 amino acids from the C-terminal residue of the transamidase recognition sequence (e.g., G). The engineered protease cleavage site may be selected in order to avoid cleavage by protease(s) found in a host cell in which the engineered precursor polypeptide is to be expressed. For example, if an engineered precursor polypeptide is to be expressed in a bacterial host cell, a protease cleavage site recognized by a mammalian endoprotease but not by bacterial proteases may be selected, and the corresponding mammalian endoprotease is then used to cleave the engineered precursor polypeptide after the engineered precursor polypeptide or multi-chain or multi-subunit protein comprising the engineered precursor polypeptide, is purified. In some embodiments of the invention a cleavage site that is cleaved by a chemical such as cyanogen bromide or hydroxylamine is used. In some embodiments the linker region of an engineered precursor polypeptide contains a cleavage site that is not otherwise present in portions of the multi-chain protein that are exposed and accessible to cleavage.

One of skill in the art will be able to select appropriate protease and chemical cleavages sites and corresponding proteases and chemical cleaving agents, respectively by referring to the literature, e.g., Keil, B. Specificity of proteolysis. Springer-Verlag Berlin-Heidelberg-NewYork, 1992 and Barrett, et al., (eds.), The Handbook of Proteolytic Enzymes, 2nd ed. Academic Press, 2003. Academic Press, 2004 and/or to databases such as MEROPS (Rawlings, N. D., et al., MEROPS: the peptidase database. Nucleic Acids Res 36, D320-D325, 2008; http://merops.sanger.ac.uk/index.htm) or the ExPASy Peptide Cutter tool available at http://www.expasy.org/tools/peptidecutter/peptidecutter_enzymes.html. These resources list numerous proteases, chemical cleaving agents, substrates, cleavage sites, and consensus cleavage sites. A protease useful in the present invention may be a serine protease, threonine protease, cysteine protease, aspartic protease, metalloprotease, or glutamic acid protease. A protease active at acid, neutral, or basic pH may be used in various embodiments of the invention.

In an exemplary embodiment, the mammalian endoprotease is trypsin (see Examples). Trypsin is a serine protease that referentially cleaves at Arg and Lys in position P1 with higher rates for Arg (Keil, 1992), especially at high pH. Pro usually blocks trypsin action when found in position P1′, with some exceptions. Other mammalian proteases of interest are factor Xa, thrombin, and enterokinase. Tobacco etch virus protease is the common name for the 27 kDa catalytic domain of the Nuclear Inclusion a (NIa) protein encoded by the tobacco etch virus (TEV). TEV protease recognizes a linear epitope of the general form E-Xaa-Xaa-Y-Xaa-Q-(G/S), with cleavage occurring between Q and G or Q and S, thus having a much more stringent sequence specificity than many other proteases. The most commonly used sequence is ENLYFQG. The following summary of the cleavage rules may be used to select a cleavage site and protease or chemical. The following enzymes potentially cleave when the respective compositions of the cleavage sites are found.

TABLE 2
Proteases, chemical cleaving agents, and cleavage sites

| Enzyme name | P4 | P3 | P2 | P1 | P1′ | P2′ |
|---|---|---|---|---|---|---|
| Arg-C proteinase | — | — | — | R | — | — |
| Asp-N | — | — | — | — | D | — |
| endopeptidase | | | | | | |
| BNPS-Skatole | — | — | — | W | — | — |
| Caspase 1 | F, W, Y, or L | — | H, A or T | D | not P, E, D, Q, | — |
| | | | | | K or R | |
| Caspase 2 | D | V | A | D | not P, E, D, Q, | — |
| | | | | | K or R | |
| Caspase 3 | D | M | Q | D | not P, E, D, Q, | — |
| | | | | | K or R | |
| Caspase 4 | L | E | V | D | not P, E, D, Q, | — |
| | | | | | K or R | |
| Caspase 5 | L or W | E | H | D | — | — |
| Caspase 6 | V | E | H or I | D | not P, E, D, Q, | — |
| | | | | | K or R | |
| Caspase 7 | D | E | V | D | not P, E, D, Q, | — |
| | | | | | K or R | |
| Caspase 8 | I or L | E | T | D | not P, E, D, Q, | — |
| | | | | | K or R | |
| Caspase 9 | L | E | H | D | — | — |
| Caspase 10 | I | E | A | D | — | — |
| Chymotrypsin-high | — | — | — | F or Y | not P | — |
| specificity (C-term to | — | — | — | W | not M or P | — |
| [FYW], not before P) | | | | | | |
| Chymotrypsin-low | — | — | — | F, L or Y | not P | — |
| specificity (C-term to | — | — | — | W | not M or P | — |
| [FYWML], not before | — | — | — | M | not P or Y | — |
| P) | — | — | — | H | not D, M, P or W | — |
| Clostripain | — | — | — | R | — | — |
| (Clostridiopeptidase | | | | | | |
| B) | | | | | | |
| CNBr | — | — | — | M | — | — |
| Enterokinase | D or N | D or N | D or N | K | — | — |
| Factor Xa | A, F, G, I, L, T, V | D or E | G | R | — | — |
| | or M | | | | | |
| Formic acid | — | — | — | D | — | — |
| Glutamyl | — | — | — | E | — | — |
| endopeptidase | | | | | | |
| GranzymeB | I | E | P | D | — | — |
| Hydroxylamine | — | — | — | N | G | — |
| Iodosobenzoic acid | — | — | — | W | — | — |
| LysC | — | — | — | K | — | — |
| NTCB (2-nitro-5- | — | — | — | — | C | — |
| thiocyanobenzoic | | | | | | |
| acid) | | | | | | |
| Pepsin (pH1.3) | — | not H, K, or R | not P | not R | F, L, W or Y | not P |
| | — | not H, K, or R | not P | F, L, W or Y | — | not P |
| Pepsin (pH > 2) | — | not H, K or R | not P | not R | F or L | not P |
| | — | not H, K or R | not P | F or L | — | not P |
| Proline- | — | — | H, K or R | P | not P | — |
| endopeptidase | | | | | | |
| Proteinase K | — | — | — | A, E, F, I, L, | — | — |
| | | | | T, V, W | | |
| | | | | or Y | | |
| Staphylococcal | — | — | not E | E | — | — |
| peptidase I | | | | | | |
| Thermolysin | — | — | — | not D or E | A, F, I, L, M or V | — |
| Thrombin | — | — | G | R | G | — |
| | A, F, G, I, L, T, V | A, F, G, I, L, T, V, | P | R | not D or E | not |
| | or M | W or A | | | | DE |
| Trypsin (please note | — | — | — | K or R | not P | — |
| exceptions below) | — | — | W | K | P | — |
| | — | — | M | R | P | — |

The above cleavage rules may not apply, i.e. cleavage may not occur, with the following compositions of the cleavage sites, so in some embodiments of the invention such sequences are not used.

| Enzyme name | P4 | P3 | P2 | P1 | P1′ | P2′ | |
|---|---|---|---|---|---|---|---|
| Trypsin | — | — | C or D | K | D | — | |
| | | — | — | C | K | H or Y | — |
| | | — | — | C | R | K | — |
| | | — | — | R | R | H or R | — |

D. Polynucleotides, Vectors, Host Cells

The invention provides polynucleotides that encode the inventive engineered precursor polypeptides. The sequences of the polynucleotides may comprise sequences as found in nature that encode the precursor polypeptide as found in nature, with appropriate modifications to encode the variants described herein. In some embodiments, the natural sequence is altered, e.g., to optimize codon usage for expression in a host cell of interest. Any nucleotide sequence may be used, provided that it encodes an inventive engineered polypeptide. The invention also provides vectors, e.g., expression vectors, in which a polynucleotide that encodes an inventive engineered precursor polypeptide is operably linked to a promoter.

Numerous promoters are known in the art and can be used. The promoter may be constitutive or inducible and may be, e.g., of viral, bacterial, fungal, plant, insect, or vertebrate origin. The invention also provides vectors that comprise a polynucleotide that encodes an inventive engineered precursor polypeptide, often operably linked to a promoter. In some embodiments the vector is a bicistronic or multi-cistronic vector. In some embodiments the vector comprises a single open reading frame (ORF) that encodes at least two distinct polypeptides (e.g., an A polypeptide and a B polypeptide of an ABₙ toxin). A single mRNA transcribed from the ORF may be translated to form two distinct polypeptides. The mRNA may comprise two or more ribosome binding sites, e.g., a Shine-Dalgarno sequence if the mRNA is to be translated in a prokaryotic host cell or a Kozak sequence or IRES if the mRNA is to be translated in a eukaryotic host cell. In some embodiments the vector comprises at least two open reading frames. A nucleic acid or vector can comprise other nucleic acid elements, e.g., regulatory elements necessary or useful for expression. For example, the nucleic acid or vector can comprise an enhancer, a polyadenylation sequence, a splice donor sequence and a splice acceptor sequence, a site for transcription initiation and termination positioned at the beginning and end, respectively, of a polypeptide to be translated, a ribosome binding site for translation in the transcribed region, an epitope tag, a nuclear localization sequence, a “TATA” element, a restriction enzyme cleavage site, a selectable marker (e.g., a nucleic acid encoding a protein that confers resistance to an antibiotic or nutritional auxotrophy, etc.). Often the nucleic acid encodes an engineered precursor polypeptide that has an N-terminal secretion signal, so that the polypeptide is secreted, e.g., into the periplasmic space of a bacterial host cell, or into the extracellular milieu. In some embodiments the secretion signal is selected to be operable in a host cell in which the polypeptide is to be expressed. For example, if the polypeptide is to be expressed in E. coli, a secretion signal from a polypeptide that is naturally expressed in and secreted by E. coli (e.g., LT) may be selected. If the polypeptide is to be expressed in yeast, a secretion signal from a polypeptide that is naturally expressed in and secreted by yeast may be selected. One of skill in the art will be able to select an appropriate promoter, other nucleic acid elements, and vector for use to express a polypeptide in a selected host cell.

The invention also provides host cells that comprise a polynucleotide or vector comprising a nucleic acid that encodes an inventive engineered precursor polypeptide. The host cell may be a prokaryotic (e.g., bacterial) or eukaryotic (e.g., fungal, plant, insect, or vertebrate (e.g., mammalian)) host cell. In some embodiments the cell is a cell of a transgenic animal or plant. Such transgenic animals or plants, which may be used to produce the inventive polypeptides and proteins, are aspects of the invention. In some embodiments the polynucleotide that encodes the inventive engineered precursor polypeptide is integrated into the chromosome of the host cell while in other embodiments it is contained in an extrachromosomal genetic element (episome) such as a plasmid. In many embodiments of the invention the host cell comprises a polynucleotide that encodes both an engineered A polypeptide of an ABₙ toxin and a native or engineered B polypeptide of an ABₙ toxin, or contains multiple polynucleotides that collectively encode both an engineered A polypeptide of an ABₙ toxin and a native or engineered B polypeptide of an ABₙ toxin, wherein the A and B polypeptides assemble to form a holotoxin. The multiple polynucleotides may be contained in a single vector or multiple vectors.

E. Methods for Producing and Sortagging Engineered Precursor Polypeptides, Multi-Chain and Multi-Subunit Proteins

An engineered precursor polypeptide of the invention may be produced by expressing a nucleic acid that encodes the polypeptide in a suitable host cell using standard methods of molecular biology. The polypeptide may be purified using methods known in the art. In some embodiments the polypeptide comprises an epitope tag to facilitate purification. Often the engineered polypeptide will be produced in a cell that also produces one or more other polypeptides that assemble together with the engineered polypeptide to form a multi-subunit protein. For example, an engineered precursor polypeptide of an A subunit of an AB₅ toxin is produced in a cell that also produces a B polypeptide. In some embodiments the multi-subunit protein assembles within the host cell and is purified therefrom. In some embodiments the multi-subunit protein assembles within the cell and is secreted therefrom and optionally purified, e.g., from culture medium. In some embodiments an engineered precursor polypeptide is chemically synthesized. However, production in host cells has certain advantages for producing multi-chain and multi-subunit proteins of the invention.

In some embodiments, cleavage occurs due to the action of a host cell protease. In other embodiments of the invention, the protein is not cleaved by a host cell protease. Instead, after an engineered precursor polypeptide or a multi-chain or multi-subunit protein comprising an engineered precursor polypeptide has been produced and, optionally purified, it may be subjected to cleavage at a cleavage site within [symbol] located C-terminal to the transamidase recognition sequence. Cleavage may be accomplished in a variety of ways. Typically, the purified protein is contacted with a suitable cleaving agent in vitro under conditions suitable for cleavage to take place. For example, cleavage may be performed by contacting the purified protein with a protease. In some embodiments of the invention the protease is immobilized (e.g., on a suitable support) thereby allowing its separation from the engineered precursor polypeptide or multi-chain or multi-subunit protein comprising the engineered precursor polypeptide following cleavage. For example, the protease could be immobilized on the walls of a tube or the bottom of a dish, on particles, rods, fibers, resins, beads (e.g., magnetic beads), etc. The cleaving conditions and agent may be selected consistent with maintaining stability of the engineered protein except with respect to the desired cleavage. After cleavage, the protease may be removed or the protein isolated from the reaction mixture in which cleavage was performed.

In the ligation methods described herein, the reaction components, e.g., a transamidase, engineered multi-chain or multi-subunit protein comprising a chain comprising a transamidase recognition sequence and the compound comprising an NH₂—CH₂— moiety, or, in other embodiments, an engineered multi-chain or multi-subunit protein comprising a chain comprising an N-terminal glycine, and a compound comprising a transamidase recognition sequence, are typically contacted with one another in a suitable receptacle or vessel to form a system. For purposes of description, the component comprising a transamidase recognition sequence (often a multi-chain or multi-subunit protein comprising a chain generated by cleavage of an engineered precursor polypeptide) is referred to herein as an acyl donor, and the nucleophilic component comprising an NH2-CH2-moiety is referred to as an acyl acceptor. Components can be contacted with one, e.g., by adding them to one body of fluid and/or placing them in one reaction vessel. The components may be mixed in a variety of ways, such as by shaking, oscillating, rotating, vortexing, rocking, repeated pipetting, or by passing fluid containing one assay component over a surface having another assay component immobilized thereon, for example. The components may typically be added in any order to the vessel but the invention encompasses embodiments in which an order is specified, e.g., the donor and acceptor are added first (in either order or a specified order) and the transamidase is added next.

A system can comprise, for example, any convenient vessel or article in which a reaction may be performed (e.g., a tube such as a microfuge tube, flask, dish), microtiter plate (e.g., 96-well or 384-well plate), etc. The system is often cell free and often does not include bacterial cell wall components or intact bacterial cell walls. In some embodiments, however, the system includes one or more cells or cell wall components. In such embodiments, one or more components, e.g., the transamidase or protein to which a compound is to be ligated) often are expressed from one or more recombinant nucleotide sequences in a cell. Cells in such systems often are maintained in suitable cell culture systems as appropriate for cells of that type.

The system comprising the reaction components is maintained at any convenient temperature at which the ligation reaction can be performed. In some embodiments, the ligation is performed at a temperature ranging from about 15° C. to about 50° C. In some embodiments, the ligation is performed at a temperature ranging from about 23° C. to about 37° C. In certain embodiments, the temperature is room temperature (e.g., about 25° C.). The temperature can be optimized by repetitively performing the same ligation procedure at different temperatures and determining ligation rates. Any convenient assay volume and component ratio is utilized. In certain embodiments, a component ratio of 1:1000 or greater transamidase enzyme to acyl donor is utilized, or a ratio of 1:1000 or greater transamidase enzyme to acyl acceptor is utilized (where a ratio is considered “greater” than 1:1000 if the second number is greater than 1000). In specific embodiments, ratios of enzyme to acyl donor or enzyme to acyl acceptor is about 1:1, including 1:2 or greater, 1:3 or greater, 1:4 or greater, 1:5 or greater, 1:6 or greater, 1:7 or greater, 1:8 or greater, 1:9 or greater, 1:10 or greater, 1:25 or greater, 1:50 or greater, or 1:100 or greater, on a molar basis.

In some embodiments, the acyl donor is present at a concentration ranging from about 10 μM to about 10 mM. In some embodiments, the acyl donor is present at a concentration ranging from about 100 μM to about 1 mM. In some embodiments, the acyl donor is present at a concentration ranging from about 200 μM to about 1 mM. In some embodiments, the acyl donor is present at a concentration ranging from about 200 μM to about 800 μM. In some embodiments, the acyl donor is present at a concentration ranging from about 400 μM to about 600 μM. In some embodiments, the nucleophilic acyl acceptor is present at a concentration ranging from about 1 μM to about 500 μM. In some embodiments, the nucleophilic acyl acceptor is present at a concentration ranging from about 15 μM to about 150 μM. In some embodiments, the nucleophilic acyl acceptor is present at a concentration ranging from about 25 μM to about 100 μM. In some embodiments, the nucleophilic acyl acceptor is present at a concentration ranging from about 40 μM to about 60 μM. In some embodiments, the transamidase is present at a concentration ranging from about 1 μM to about 500 μM. In some embodiments, the transamidase is present at a concentration ranging from about 15 μM to about 150 μM. In some embodiments, the transamidase is present at a concentration ranging from about 25 μM to about 100 μM. In some embodiments, the transamidase is present at a concentration ranging from about 40 μM to about 60 μM.

In some embodiments, the ligation method is performed in a system comprising an aqueous environment. Water with an appropriate buffer and/or salt content is often utilized. An alcohol or organic solvent may be included in certain embodiments. The amount of an organic solvent often does not appreciably esterify a protein or peptide in the ligation process (e.g., esterified protein or peptide often increase only by 5% or less upon addition of an alcohol or organic solvent). Alcohol and/or organic solvent contents sometimes are 20% or less, 15% or less, 10% or less or 5% or less, and in embodiments where a greater amount of an alcohol or organic solvent is utilized, 30% or less, 40% or less, 50% or less, 60% or less, 70% or less, or 80% or less alcohol or organic solvent is present. In certain embodiments, the system includes only an alcohol or an organic solvent, with only limited amounts of water if it is present.

In some embodiments, suitable ligation conditions comprise a buffer. One of ordinary skill in the art will be familiar with a variety of buffers that could be used in the present invention. In some embodiments, the buffer solution comprises calcium ions. In certain embodiments, the buffer solution does not contain substances that precipitate calcium ions. In some embodiments, the buffer solution does not include phosphate ions. In some embodiments, the buffer solution does not contain chelating agents.

In some embodiments, suitable ligation conditions comprise pH in the range of 6 to 8.5. In some embodiments, suitable ligation conditions comprise pH in the range of 6 to 8. In some embodiments, suitable ligation conditions comprise pH in the range of 6 to 7.5. In some embodiments, suitable ligation conditions comprise pH in the range of 6.5 to 8.5. In some embodiments, suitable ligation conditions comprise pH in the range of 7 to 8.5. In some embodiments, suitable ligation conditions comprise pH in the range of 7.5 to 8.5. In some embodiments, suitable ligation conditions comprise pH in the range of 7.0 to 8.5. In some embodiments, suitable ligation conditions comprise pH in the range of 7.3 to 7.8.

One or more components for ligation or a ligation product may be immobilized to a solid support. The attachment between an assay component and the solid support may be covalent or non-covalent (e.g., U.S. Pat. No. 6,022,688 for non-covalent attachments). The solid support may be one or more surfaces of the system, such as one or more surfaces in each well of a microtiter plate, a surface of a glass slide or silicon wafer, Biacore chip, a surface of a particle, e.g., a bead, that is optionally linked to another solid support, or a channel in a microfluidic device, for example. Types of solid supports, linker molecules for covalent and non-covalent attachments to solid supports, and methods for immobilizing molecules to solid supports are known (e.g., U.S. Pat. Nos. 6,261,776; 5,900,481; 6,133,436; and 6,022,688; and WIPO publication WO 01/18234). In some embodiments a reaction component is immobilized by adsorption. A support can be made out of a wide variety of organic or inorganic materials or mixtures thereof and can have a variety of different shapes and sizes. Exemplary materials that may be used in the manufacture of suitable vessels or supports are polymeric materials, e.g., plastics, such as polypropylene, polystyrene, poly(meth)acrylates, polybutadienes, and the like, individually or in the form of copolymers or blends, other polymers such as cellulose, etc. Exemplary inorganic materials are silicon oxide, silicon, mica, glass, quartz, titanium oxide, vanadium oxide, metals such as gold or silver, alloys such as steel, etc. In some embodiments the solid support is semi-solid and/or gel-like, deformable, flexible, or the like. For example a semisolid material such as a gel (e.g., formed at least in part from organic polymers such as PDMS), etc. or agarose may be used. The system can include ancillary equipment such as robotic platforms, liquid dispensers, and signal detectors.

In some embodiments, after the ligation has been performed, the modified multi-chain or multi-subunit protein is separated from the transamidase and, optionally, other reaction components. Any suitable means for separation or purification may be used. For example, such separation may be based on molecular weight, affinity approaches, dialysis using appropriate membranes, or combinations of such approaches, etc. In some embodiments, a purification tag is used. The tag may if desired be removed, e.g., by cleavage, after purification of the protein.

### III. Compounds of Interest and Applications for Modified Multi-Chain and Multi-Subunit Proteins

A wide variety of compounds of interest can be attached to a polypeptide or multi-chain or multi-subunit protein using the inventive methods, and the resulting modified polypeptides, multi-chain and multi-subunit proteins have a variety of uses that depend at least in part on the identity of the compound of interest. An application of particular note is the use of a multi-chain or multi-subunit protein to deliver a compound of interest to the cytoplasm of a eukaryotic cell, e.g., a mammalian cell. In some embodiments the mammalian cell is a human cell. The compound of interest may be, e.g., a therapeutic agent or an antigen. If the compound of interest comprises an antigen, the modified multi-chain or multi-subunit protein may serve as a component of a vaccine. For example, the modified protein may be combined with a pharmacologically acceptable carrier to form a vaccine that may be administered to a subject, e.g., a mammal, to generate immunological protection against a wide variety of pathogens or to provoke an immunological response against deleterious “self” cells, e.g., cancer cells, or other self cells whose presence contributes to a disease or other an undesirable condition.

A compound to be ligated to a polypeptide comprising a transamidase recognition sequence according to the present invention typically comprises an NH₂—CH₂— moiety, e.g., NH₂—CH₂(C═O)—Z¹. In some embodiments compound has formula (G)ₖ-Z¹, wherein Z¹ is or comprises acyl, substituted or unsubstituted aliphatic, substituted or unsubstituted heteroaliphatic, substituted or unsubstituted aryl, substituted or unsubstituted heteroaryl, a peptide, a protein, a polynucleotide, a sugar, a tag, a metal atom, a contrast agent, a catalyst, a non-polypeptide polymer, a specific binding pair member, a cross-linkable moiety, a small molecule, a lipid, a photoaffinity probe, a particle, or a label; G is glycine; and k is an integer from 1 to 6, inclusive. In those embodiments in which a compound is to be ligated to a polypeptide comprising an N-terminal G residue, the compound can have formula transamidase recognition sequence—Z¹, where Z¹ is as indicated above. In some embodiments, Z¹ comprises a polypeptide no longer than 300 amino acids, in some embodiments no longer than 250 amino acids, in some embodiments no longer than 200 amino acids, in some embodiments no longer than 150 amino acids, in some embodiments between 100 and 150 amino acids, in some embodiments between 50 and 100 amino acids, in length. In some embodiments, Z¹ has a molecular weight no more than 5, 10, 20, 30, 40, or 50 kD. In some embodiments, Z¹ comprises an antigen or therapeutic agent, examples of which are discussed below. In some embodiments a label comprises a fluorescent label, a radiolabel, a chemiluminescent label, or a phosphorescent label. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioisotopes include ¹²⁵I, ¹³¹I, ³⁵S or ³H. The radioisotope sometimes is selected based upon its appropriate use in a nuclear medicinal procedure, such as Be-7, Mg-28, Co-57, Zn-65, Cu-67, Ge-68, Sr-82, Rb-83, Tc-95m, Tc-96, Pd-103, Cd-109, and Xe-127, to name but a few. In some embodiments a particle comprises a metal (e.g., gold), a quantum dot, a polymer, or a label. In some embodiments a polymer is a nanoparticle (having a diameter less than 1000 nm). In some embodiments a particle is a microparticle (having a diameter of 1000 nm or more but less than 500 microns). In some embodiments a specific binding pair member is a compound that binds specifically to a second compound, e.g., a polypeptide comprising an antigen-binding portion of an antibody, biotin, streptavidin/avidin, etc.). In some embodiments a particle is a liposome or other lipid-based particle. In some embodiments the particle comprises at least 50% lipids by dry weight. The lipid-based particle may comprise phospholipids, e.g., phosphatidylethanolamine, surfactant components such as dioleoylphosphatidylethanolamine, and other components known in the art. See, e.g., Liposomes, Parts A, B, C, and D, Methods in Enzymology (vols. 367, 372, 373, and 387), Academic Press. For example, in some embodiments the liposomes contains a core comprising an aqueous solution. In some embodiments the particle comprises a compound. In general, the compound may be acyl, substituted or unsubstituted aliphatic, substituted or unsubstituted heteroaliphatic, substituted or unsubstituted aryl, substituted or unsubstituted heteroaryl, a peptide, a protein, a polynucleotide, a sugar, a metal atom, a contrast agent, a catalyst, a non-polypeptide polymer, a specific binding pair member, a cross-linkable moiety, a small molecule, a lipid, a photoaffinity probe, or a label. In some embodiments the particle comprises an antigen or a therapeutic agent. A polynucleotide can be single-stranded, double-stranded, or partly single and partly double-stranded. It can be a short interfering RNA (siRNA), microRNA, ribozyme, antisense molecule, or aptamer. A polypeptide or peptide can be linear, branched, or cyclic. The polypeptide can be a glycoprotein, lipoprotein, phosphoprotein, or have any other modification. In some embodiments Z¹ comprises an enzyme. The enzyme may be, e.g., an oxidoreductase, a transferases, hydrolase, a lyases, an isomerase, or a ligase. In some embodiments the enzyme is a protease, lipase, endonuclease, exonuclease, polymerase, recombinase, kinase, phosphatase, or GTPase. For example, the enzyme may be Cre recombinase. In some embodiments Z¹ comprises an enzyme inhibitor. The inhibitor may inhibit an enzyme of any of the afore-mentioned types. In some embodiments the compound of interest comprises an antibody or antibody fragment or antigen-binding domain of an immunoglobulin. Antibodies or purified fragments having an antigen binding domain can be fragments such as Fv, Fab′, F(ab′)2, single chain antibodies (which include the variable regions of the heavy and light chains of an immunoglobulin, linked together with a short linker), or complementarily determining regions (CDRs). In other embodiments the compound of interest does not comprise an antibody or antibody fragment or antigen-binding domain of an immunoglobulin. In most embodiments the compound of interest does not comprise the Ig-binding D region (DD) of staphylococcal A protein (Ljungberg, U K, et al., Mol Immunol. 30:1279, 1993; Agren L, et al., J Immunol. 164(12):6276-86, 2000).

In some embodiments, Z¹ comprises a subcellular targeting moiety or “sorting signal”. The subcellular targeting moiety can be a peptide domain used by a cell to target a protein to an organelle such as the nucleus, mitochondria, or peroxisome. The subcellular targeting moiety can be selected to be functional in a cell type to which an inventive modified ABₙ toxin is to be delivered, e.g., a mammalian cell. One of skill in the art will be aware of suitable subcellular targeting moieties.

In embodiments in which Z¹ is a polypeptide, the compound can be produced using standard chemical synthesis methods or using recombinant DNA technology as known in the art. For example, a peptide or polypeptide comprising one or more glycine residues at its N terminus can be chemically synthesized using standard solid phase peptide synthesis or produced as a fusion protein. In embodiments in which Z¹ is or comprises a non-polypeptide moiety, a variety of methods may be used to prepare the compound. In some embodiments the compound is chemically synthesized. In some embodiments, Z¹ comprises (i) a peptide moiety, e.g., (G)ₖ, where k is an integer between 1 and 6, e.g., between 3 and 5, and (ii) a non-polypeptide moiety such as a lipid, nucleic acid, carbohydrate, non-peptidic small molecule, etc. In such embodiments a variety of methods may be used to attach the non-polypeptide moiety to the peptide moiety. Methods for covalently or noncovalently linking moieties are known in the art and need not be described in detail here. General methods for conjugation and cross-linking are described in “Cross-Linking”, Pierce Chemical Technical Library, available at the Web site having URL www.piercenet.com and originally published in the 1994-95 Pierce Catalog and references cited therein, in Wong S S, Chemistry of Protein Conjugation and Crosslinking, CRC Press Publishers, Boca Raton, 1991; and G. T. Hermanson, Bioconjuate Techniques, 2^{nd} ed. Academic Press, 2008. For example, according to certain embodiments of the invention a bifunctional crosslinking reagent is used to couple a non-polypeptide moiety to a peptide that comprises a (G)ₖ moiety. In general, bifunctional crosslinking reagents contain two reactive groups, thereby providing a means of covalently linking two target groups. The reactive groups in a chemical crosslinking reagent typically belong to various classes including succinimidyl esters, maleimides, pyridyldisulfides, and iodoacetamides. In some embodiments, a non-polypeptide moiety is linked to the C-terminus of a peptide comprising (G)ₖ. In other embodiments a non-polypeptide moiety is linked to a side chain of a peptide comprising (G)ₖ. The peptide may contain an amino acid selected to facilitate convenient modification, e.g., a lysine residue.

In some embodiments Z¹ comprises two or more moieties. The two or more moieties may be covalently or noncovalently attached to one another or to a third moiety. For example, Z¹ can comprise a peptide, wherein a first moiety is attached to a side chain of a lysine residue in the peptide and a second moiety attached at the C-terminal end of the peptide. For example, Z¹ could comprise a label (e.g., a fluorophore) and a therapeutic agent or antigen. The label is used to monitor delivery of Z¹ to the cytosol (or to an intracellular compartment). In another embodiment, Z¹ comprises multiple different antigens or multiple “copies” of the same antigen. In another embodiment, Z¹ comprises an antigenic peptide and has a particle attached thereto. The particle may, e.g., comprise a therapeutic agent.

A. Antigens and Immunogenic Compositions

In certain embodiments, the compound of interest to be attached to an engineered polypeptide (e.g., an A1 chain of an AB5 toxin) comprises an antigen. The invention provides immunogenic compositions comprising a modified AB₅ toxin protein, wherein an antigen is attached to the A1 chain of the toxin protein. In some embodiments the antigen is attached according to the inventive transamidase-mediated ligation method of the invention. The immunogenic composition (also referred to as a “vaccine composition”) may be used to generate or stimulate an immune response ex vivo or in vivo. In various embodiments of the invention the composition may be used to generate or stimulate an immune response prophylactically (i.e., before infection or development of an undesirable condition such as a tumor or before symptoms thereof are evident) or may be administered after infection or development of an undesirable condition or symptoms thereof are evident.

In some embodiments an immunogenic composition of the invention provides protection against an infection or other disorder that affects an organ having a mucosal surface. In some embodiments an immunogenic composition of the invention protects against a pathogen characterized in that infection affects or starts from a mucosal surface. In some embodiments the vaccine composition provides protection against an enteric infection such as infection by V. cholerae, S. typhi, enterotoxigenic E. coli (ETEC), Shigella spp, C. difficile, rotavirus, calicivirus. In some embodiments the vaccine composition provides protection against an infection affecting the respiratory system such as M. pneumoniae, influenza virus, or respiratory syncitial virus. In some embodiments the vaccine composition provides protection against a sexually transmitted infection such as infection with HIV, herpes simplex virus, C. trachomatis, or N. gonorrhoeae.

The antigen may be any molecule or portion thereof recognized by the immune system of a subject as foreign. In some embodiment, the antigen is a substance that stimulates or enhances an immune response, following exposure to or contact with the antigen. An antigen may be a protein, a glycoprotein, a nucleic acid, a carbohydrate, a proteoglycan, a lipid, a mucin molecule, or other similar molecule, including any combination thereof. In some embodiments the antigen is or comprises a peptide. The peptide may be, e.g., between 6 and 20 amino acids long, e.g., 8, 9, 10, 11, or 12 amino acids long. The antigen may, in another embodiment, be a cell or a part thereof, for example, a cell surface molecule, cell wall component, etc. In some embodiments, the antigen may be derived from an infectious or pathogenic virus, bacterium, fungus, parasite, etc., or part thereof. The infectious organism may be virulent, in some embodiments or avirulent, in other embodiments. An organism may be rendered avirulent, for example, by exposure to heat, chemical treatment (e.g., formaldehyde), or removal of at least one protein or gene required for replication of the organism. In some embodiments, an antigenic protein or peptide is isolated (e.g., from cells that naturally produce it or are engineered to produce it), or in another embodiment, synthesized. In some embodiments, the antigen is derived from a neoplastic or preneoplastic cell. In some embodiment, the antigen is an autoantigen, or a molecule which initiates or enhances an autoimmune response. In certain embodiments an antigen is a peptide whose sequence is found in a polypeptide expressed by a pathogen or tumor.

In some embodiments the antigen is derived from an infectious virus such as, e.g., a member of the family Retroviridae or Lentiviridae (e.g. human immunodeficiency viruses, such as HIV-I, HIV-II, HTLV-I, HTLV-II, etc.); Picornaviridae (e.g. polio viruses, hepatitis A virus; enteroviruses, human coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronaviridae (e.g. coronaviruses); Rhabdoviridae (e.g, vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxyiridae (variola viruses, vaccinia viruses, pox viruses); and Iridoviridae (e.g. African swine fever virus); the agent of delta hepatitis, Hepatitis C virus; Norwalk and related viruses, and astro viruses. Without limitation, the antigen may be derived from Respiratory syncytial virus, Parainfluenza virus types 1-3, Human metapneumovirus, Influenza virus, Herpes simplex virus, Human cytomegalovirus, Human immunodeficiency virus, Simian immunodeficiency virus, Hepatitis A virus, Hepatitis B virus, Hepatitis C virus, Human papillomavirus, Poliovirus, rotavirus, caliciviruses, Measles virus, Mumps virus, Rubella virus, rhinovirus, calicivirus, adenovirus, rabies virus, canine distemper virus, rinderpest virus, avian pneumovirus, Ebola virus, Marburg virus, hantavirus, Hendra virus, Nipah virus, coronavirus, parvovirus, infectious rhinotracheitis viruses, feline leukemia virus, feline infectious peritonitis virus, avian infectious bursal disease virus, Newcastle disease virus, Marek's disease virus, porcine respiratory and reproductive syndrome virus, equine arteritis virus, foot-and-mouth disease virus, and encephalitis viruses. In some embodiments the pathogenic virus infects human hosts. In some embodiments the pathogenic virus infects non-human animals, e.g., swine, ovines, bovines, canines, felines, avians, etc.

In some embodiments the antigen is derived from a bacterium such as, e.g., Helicobacter pylori, Boreilia burgdorferi, Legionella pneumophilia, Mycobacteria sps (e.g. M. tuberculosis, M. avium, M, intracellulars M. kansaii, M. gordonae), Staphylococcus aureus, Staphylococcus epidermidis, Neisseria gonorrhoeae, Neisseria meningitidis (e.g, of serogroup A, B, C, Y, or W135), Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Chlamydia sp., Haemophilus influenzae, Haemophilus somnus, Bacillus antracis, Corynebacterium diphtheriae, corynebacterium sp., Erysipelothrix rhusiopathiae, Clostridium perfringens, Clostridium tetani, Enterobacter aerogenes, Klebsiella pneumoniae, Pasturella inultocida, Bacteroides sp., Fusobacterium nucleatum, Streptobacillus moniliformis, Treponema pallidium, Treponema pertenue, Leptospira, Actinomyces israelii, Francisella tularensis, Haemophilus somnus, Moraxella catarrhalis, Chlamydia trachoniatis, Chlamydia pneumoniae, Chlamydia psittaci, Bordetella pertussis, Alloiococcus otiditis, Salmonella typhi, Salmonella typhimurium, Salmonella choleraesuis, Escherichia coli (e.g., pathogenic E. coli), Shigella, Vibrio cholerae, Corynebacterium diphtheriae, Mycobacterium tuberculosis, Mycobacterium avium-Mycobacterium intracellulare complex, Proteus mirabilis, Proteus vulgaris, Pseudomonas, Klebsiella, Clostridium tetani, C. difficile, Leptospira, Legionella, Listeria, Borrelia burgdorferi, Brucella abortus, Pasteurella haemolytica, Pasteurella multocida, Actinobacillus pleuropneumoniae and Mycoplasma gallisepticum, or any other bacterium within the same genus as one or more of the foregoing. In some embodiments the pathogenic bacterium infects human hosts. In some embodiments the pathogenic bacterium infects non-human animals.

In some embodiments, the antigen is derived from a fungus such as, e.g., Absidia, such as Absidia corymbifera, Ajellomyces, such as Ajellomyces capsulatus, Ajellomyces dermatitidis, Arthroderma, such as Arthroderma benhamiae, Arthroderma fulvum, Arthroderma gypseum, Arthroderma incurvatum, Arthroderma otae, Arthroderma vanbreuseghemii, Aspergillus, such as Aspergillus flavus, Aspergillus fumigatus, Aspergillus niger, Blastomyces, such as Blastomyces dermatitidis, Candida, such as Candida albicans, Candida glabrata, Candida guilliermondii, Candida krusei, Candida parapsilosis, Candida tropicalis, Candida pelliculosa Cladophialophora, such as Cladophialophora carrionii, Coccidioides, such as Coccidioides immitis, Cryptococcus, such as Cryptococcus neoformans, Cunninghamella, Epidermophyton, such as Epidermophyton floccosum, Exophiala, such Exophiala dermatitidis, Filobasidiella, such as Filobasidiella neoformans, Fonsecaea, such as Fonsecaea pedrosoi, Fusarium, such as Fusarium solani, Geotrichum, such as Geotrichum candidum, Histoplasma, such as Histoplasma capsulatum, Hortaea, such as Hortaea werneckii, Issatschenkia, such as Issatschenkia orientalis, Madurella, such Madurella grisae, Malassezia, such as Malassezia furfur, Malassezia globosa, Malassezia obtuse, Malassezia pachydermatis, Malassezia restricta, Malassezia slooffiae, Malassezia sympodialis, Microsporum, such as Microsporum canis, Microsporum fulvum, Microsporum gypseum, Mucor, such as Mucor circinelloides, Nectria, such as Nectria haematococca, Paecilomyces, such as Paecilomyces variotii, Paracoccidioides, such as Paracoccidioides brasiliensis, Penicillium, such as Penicillium marneffei, Pichia, such as Pichia anomala, Pichia guilliermondii, Pneumocystis, such as Pneumocystis carinii, Pseudallescheria, such as Pseudallescheria boydii, Rhizopus, such as Rhizopus oryzae, Rhodotorula, such as Rhodotorula rubra, Scedosporium, such as Scedosporium apiospermum, Schizophyllum, such as Schizophyllum commune, Sporothrix, such as Sporothrix schenckii, Trichophyton, such as Trichophyton mentagrophytes, Trichophyton rubrum, Trichophyton verrucosum, Trichophyton violaceutn, Trichosporon, such as Trichosporon asahii, Trichosporon cutaneum, Trichosporon inkin, Trichosporon mucoides, or others. In some embodiments the pathogenic fungus infects human hosts. In some embodiments the pathogenic fungus infects non-human animals.

In some embodiments the antigen is derived from a parasitic organism. In some embodiments the organism is one that resides intracellularly during at least some stages of its life cycle. Parasites contemplated include for example, parasites of the genus Plasmodium (e.g. Plasmodium falciparum, P. vivax, P. ovale and P. malariae), Trypanosoma, Toxoplasma (e.g., Toxoplasma gondii), Leishmania (e.g., Leishmania major), Schistosoma, and Cryptosporidium Pneumocystis carinii. In some embodiments the parasitic agent resides extracellularly during at least part of its life cycle. Examples include nematodes, trematodes (flukes), and cestodes. Without limitation, antigens from Ascaris or Trichuris are contemplated. In some embodiments, the antigen is derived from a byproduct of infection with the parasite, for example, egg antigens of Schistosoma, antigens uniquely expressed in Toxoplasma cysts, etc., as will be appreciated by one skilled in the art. In some embodiments the pathogenic parasite infects human hosts. In some embodiments the pathogenic parasite infects non-human animals.

In some embodiments, the antigen is derived from a diseased, abnormal, and/or undesired cell. The diseased, abnormal, or undersired cells contemplated include: infected cells, tumor cells, self-reactive cells, e.g., self-reactive T cells and plasma cells that produce auto-antibodies. In some embodiments the diseased, abnormal, or undesired cells are obtained from a subject and used to prepare an antigen, which is used to prepare an immunogenic composition of the invention. The composition is administered to the subject from which the cells were obtained or to a different subject suffering from the same or a similar disease or condition.

In some embodiments, the antigen is a tumor-associated antigen, e.g., a molecule that is expressed selectively or specifically by tumor cells. The term “tumor” is intended to encompass benign tumors, premalignant tumors, and malignant tumors, i.e., cancers. A cancer may be a carcinoma (a malignant tumor derived from epithelial cells such as the common forms of breast, prostate, lung and colon cancer), a sarcoma (a malignant tumor derived from connective tissue, or mesenchymal cells), a lymphoma or leukemia (malignancies derived from hematopoietic cells, or a germ cell tumor (derived from totipotent cells). In some embodiments the tumor is one that resembles an immature or embryonic tissue.

A variety of tumor-associated antigens are known in the art and are of use in embodiments of the invention. Examples are the KS 1/4 pan-carcinoma antigen (Perez and Walker, 1990, J. Immunol. 142:32-37; Bumal, 1988, Hybridoma 7(4):407-415), CA125, often associated with ovarian cancer (Yu et al, 1991, Cancer Res. 51(2):48-475), prostatic acid phosphate (Tailor et al, 1990, Nucl. Acids Res. 18(1):4928), prostate specific antigen (Henttu and Vihko, 1989, Biochem. Biophys. Res. Comm. 10(2):903-910; Israeli et al, 1993, Cancer Res. 53:227-230), melanoma-associated antigen p 97 (Estin et al, 1989, J. Natl. Cancer Instit. 81 (6):445-44), melanoma antigen gp75 (Vijayasardahl et al, 1990, J. Exp. Med. 171(4):1375-1380), high molecular weight melanoma antigen (HMW-MAA) (Natali et al, 1987, Cancer 59:55-3; Mittelman et al, 1990, J. Clin. Invest. 86:2136-2144)), prostate specific membrane antigen, carcinoembryonic antigen (CEA), often associated with colorectal cancer (Foon et al, 1994, Proc. Am. Soc. Clin. Oncol. 13:294), TAG-72 (Yokata et al, 1992, Cancer Res. 52:3402-3408), CO17-1A (Ragnhammar et al, 1993, Int. J. Cancer 53:751-758); GICA 19-9 (Herlyn et al, 1982, J. Clin. Immunol. 2:135), CTA-I and LEA, Burkitt's lymphoma antigen-38.13, CD19 (Ghetie et al, 1994, Blood 83: 1329-1336), human B-lymphoma antigen-CD20 (Reffef al, 1994, Blood 83:435-445), CD33 (Sgouros et al, 1993, J. Nucl. Med. 34:422-430), melanoma-specific antigens such as ganglioside GD2 (Saleh et al, 1993, J. Immunol., 151, 3390-3398), ganglioside GD3 (Shitara et al, 1993, Cancer Immunol. Immunother. 36:373-380), ganglioside GM2 (Livingston et al, 1994, J. Clin. Oncol. 12: 1036-1044), tumor-specific transplantation type of cell-surface antigen (TSTA) such as virally-induced tumor-associated antigens including T-antigen DNA tumor viruses and envelope antigens of RNA tumor viruses, carcinoembryonic antigen such as CEA (Hellstrom et al, 1985, Cancer. Res. 45:2210-2188), differentiation antigen such as human lung carcinoma antigen L6, L20 (Hellstrom et al, 1986, Cancer Res. 46:3917-3923), antigens of fibrosarcoma, human leukemia T cell antigen-Gp37 (Bhattacharya-Chatterjee et al, 1988, J. of Immun. 141:1398-1403), an antigen such as EGFR (Epidermal growth factor receptor), HER2 antigen (p185HER2) associated with breast cancer, etc. In some embodiments the tumor-associated antigen is from a brain tumor, e.g., a glioma, a glioblastoma, a gliosarcoma, an astrocytoma. In some embodiments, the antigen is derived from HER2/neu or carcinoembryonic antigen (CEA). Without limitation, a vaccine comprising such antigen may be of use for suppression of cancers of the breast, ovary, pancreas, colon, prostate, and lung, which express these antigens. Similarly, mucin-type antigens such as MUC-1 can be used against various carcinomas; the MAGE, BAGE, and Mart-1 antigens can be used against melanomas. In some embodiments, the methods may be tailored to a specific cancer patient, such that the choice of antigenic peptide or protein is based on which antigen(s) are expressed in the patient's cancer cells, which may be determined, e.g., by analyzing cells obtained from the cancer or by using such cells to prepare the antigen. It will be appreciated that many antigens are expressed by more than one type of tumor and the identification of particular antigens with certain tumor types above is not intended to limit the uses of the invention to those particular tumor types but represent exemplary tumors that may be treated using the inventive immunomodulating compositions.

In some embodiments an antigen is derived from an oncoprotein of an oncogenic virus, e.g., a papilloma virus. For example, an antigen may be derived from the E6 or E7 oncoprotein from human papillomavirus 16 (HPV16) (see Example 4).

In some embodiments an antigen is derived from a molecule that is expressed by rapidly dividing cells or is required for cell immortalization. In some embodiments an antigen is found in multiple different tumor types. In some embodiments an antigen is a peptide derived from hTERT. See, e.g., WO/2000/025813 (PCT/US1999/025438) for discussion of antigens derived from hTERT and other information that may be applied in the context of the invention. In some embodiments an antigen is derived from a mutant form of a protein, e.g., an oncoprotein, that is not derived from an oncogenic virus. The antigen could comprise, for example, a portion of the protein that differs from its normal, non-oncogenic counterpart. In some embodiments the antigen is derived from a protein or portion thereof that is present on the cell surface of tumor cells, e.g., an extracellular portion of a receptor.

In some embodiments, the antigen is an endogenous protein associated with disease. Aggregated or misfolded proteins play a role in the pathogenesis of a number of diseases, e.g., amyloid beta (Abeta) in Alzheimer's disease, PrP or other prion proteins in spongiform encephalopathies, and a variety of other proteins involved in amyloidoses. In some embodiments, an antigen is derived from such a disease-associated protein.

In some embodiments, the antigen is an endogenous (“self”) protein or other self molecule associated with autoimmune disease. For example, the antigen may be derived from myelin basic protein, associated with multiple sclerosis. In other embodiments the antigen may be derived from a molecule associated with type I diabetes, Behcet's disease (e.g., human heat shock 60 protein), scleroderma, ankylosing spondylitis, sarcoid, pemphigus vulgaris, myasthenia gravis (e.g., acetylcholine receptor (AChR)), systemic lupus erythemotasus, rheumatoid arthritis, juvenile arthritis, Reiter's disease, Berger's disease, dermatomyositis, Wegener's granulomatosis, autoimmune myocarditis, anti-glomerular basement membrane disease (e.g., Goodpasture's syndrome), dilated cardiomyopathy, thyroiditis (e.g., Hashimoto's thyroiditis, Graves' disease), or Guillane-Barre syndrome. Administration, e.g., oral or nasal administration, of an inventive modified ABₙ toxin may be used to induce tolerance to such self antigen(s).

In other embodiments, the antigen is a substance capable of stimulating a hypersensitivity reaction in a mammal, e.g., a type-I or type-IV hypersensitivity reaction. For example, the antigen may be a substance capable of causing an allergy in an atopic individual. In some embodiments an antigen is derived from a food substance (e.g., dairy, nut (e.g., peanut), soy, wheat, egg, or shellfish). In some embodiments an antigen is a substance present in the environment, e.g., dog or cat dander, dust mites, mold, or pollen. In some embodiments an antigen is a substance capable of causing an asthmatic attack in an individual suffering from asthma. Administration, e.g., oral or nasal administration, of an inventive modified ABₙ toxin may be used to induce tolerance to such environmental antigen(s).

It will be understood that an antigen “derived from” a particular naturally occurring molecule may be produced using any suitable means and need not be obtained from the source in which it occurs in nature, though in some embodiments the antigen is obtained from such source. For example, antigens can be chemically synthesized, produced using recombinant DNA technology, etc. Antigens can also be modified, combined, conjugated to one another or to a carrier, etc. In some embodiments, antigens comprise additional elements not present in a naturally occurring molecule from which the antigen is derived. For example, a peptide may be extended at either end. In some embodiments, an antigen differs from a naturally occurring molecule from which the antigen is derived. For example, a peptide may have one or more substitutions or deletions. In some embodiments, multiple peptide antigens are combined to form a longer polypeptide, which is attached to an A1 chain. Such antigens could be derived from a single infectious agent, tumor, etc., or could be derived from different infectious agents, tumors, etc.

In some embodiments the antigen comprises at least one T cell epitope, e.g., a CD8+ T cell epitope.

Without wishing to be bound by any theory, the compositions and methods of the invention offer a number of advantages for vaccine preparation. Certain embodiments of the inventive approach provide both the adjuvant effect of an AB₅ toxin as well as the ability to deliver an antigen of interest to the cytoplasm.

Certain pathogens mutate rapidly and/or undergo frequent mixing or reassortment of segments of their genome. Influenza virus (e.g., influenza A virus) is a notable example. Each year a prediction is made regarding which strains are likely to be circulating, and vaccines comprising live (attenuated) or inactivated viruses are produced for that year. According to certain aspects of the present invention, an engineered AB₅ toxin is prepared and stored (e.g., for 3-6 months, or longer). Upon predicting which strains are likely to be prevalent in any given year, the engineered AB₅ toxin is modified by ligating appropriate antigen(s) corresponding to the particular strains against which immunity is sought. For example, if an H5N1 strain is expected to be prevalent, antigens, e.g., peptides, from the H5 or N1 polypeptides may be used. In another embodiment, a preparation of previously produced engineered AB₅ toxin is used to rapidly prepare a vaccine composition to be used to confer protection against a newly or recently identified pathogen (e.g., a newly identified virus such as the causative agent of SARS). In some embodiments an engineered AB₅ toxin is used to prepare a vaccine against a pathogen against which it has not previously been possible to develop a safe and effective vaccine.

The invention also provides compositions comprising: (i) a modified engineered polypeptide, multi-chain protein, or multi-subunit protein of the invention, e.g., a modified AB₅ toxin having a compound of interest, e.g., an antigen, attached to the A1 chain; and (ii) an immunomodulating compound. The invention also provides methods in which a modified engineered polypeptide, multi-chain protein, or multi-subunit protein of the invention, e.g., a modified AB₅ toxin having a compound of interest, e.g., an antigen, attached to the A1 chain is used in combination with an immunomodulating compound, e.g., to contact a cell or treat a subject. An immunomodulating compound may be an immunostimulating compound. Examples of useful immunomodulating proteins include cytokines, chemokines, complement components, immune system accessory and adhesion molecules and their receptors of human or non-human animal specificity. See, e.g., Paul, W E (ed.), Fundamental Immunology, Lippincott Williams & Wilkins; 6th ed., 2008. Useful examples include, but are not limited to: interleukins for example interleukins 1 to 15, interferons alpha, beta or gamma, tumor necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIF-Ia and MIP-Ib. In some embodiments an immunomodulating compound is a Toll-like receptor (TLR) ligand, e.g., a TLR agonist. For example, the TLR ligand may be a ligand of any TLR (e.g., TLR1-13). In some embodiments the TLR is a TLR found in humans. Exemplary TLR ligands include, e.g., dsRNA (e.g., of viruses), unmethylated CpG, bacterial lipopolysaccharides (LPS), proteins such as flagellin from bacterial flagella etc. In some embodiments the TLR ligand is a TLR3 ligand. In some embodiments the TLR ligand is a TLR4 ligand. In some embodiments the TLR ligand is a TLR9 ligand.

B. Therapeutic Agents

In some embodiments a compound of interest comprises a therapeutic agent that produces a beneficial effect through a mechanism other than serving as an antigen to produce or enhance an immune response. In some embodiments of the invention the compound of interest comprises a therapeutic agent that is of use to treat a disease or clinical condition and acts at least in part by a mechanism other than by producing or enhancing an immune response. Often the therapeutic agent is a compound that binds to an endogenous cellular protein or nucleic acid, or complex comprising protein(s) and/or nucleic acids, found in a cell that expresses a receptor for the modified AB5 toxin. Often the therapeutic agent is a compound that binds to an endogenous cellular protein or nucleic acid in the cytoplasm or nucleus of the cell. Exemplary agents may be proteins, peptides, nucleic acids (e.g., siRNAs, microRNAs, antisense oligonucleotides, antagomirs, aptamers, etc.), or small molecules. The therapeutic agent could fall into any chemical class or mechanistic category and could be useful to treat any disease of interest. In some embodiments the agent is one that does not readily cross the plasma membrane of a mammalian cell in the absence of a delivery agent. One of skill in the art will be aware of numerous therapeutic agents and diseases that may be treated using them. See, e.g., Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 9th edition (December 2003); Goldman & Ausiello, Cecil Textbook of Medicine, 22nd ed., W.B. Saunders, 2003.

C. Formulations and Administration

In some embodiments of the invention an engineered AB₅ toxin of the invention is used to prepare a suitable pharmaceutical or vaccine composition. Such compositions are aspects of this invention. The composition can be prepared using methods known in the art. The engineered AB5 toxin is typically combined with an immunologically acceptable diluent or a pharmaceutically acceptable carrier, such as sterile water or sterile isotonic saline. The modified proteins may be mixed with such diluents or carriers in a conventional manner. As used herein the language “pharmaceutically acceptable carrier” is intended to include any and all solvents, dispersion media, coatings, antibacterial and antifungal agents, isotonic and absorption delaying agents, and the like, compatible with administration to humans or other vertebrates. An appropriate carrier will be evident to those skilled in the art and will depend in large part upon the route of administration. The composition may be substantially free of endotoxin or other undesirable substances and suitable for administration to humans or animals. In some embodiments the composition is substantially free of components, e.g., transamidase, protease, or other reagents used in producing the modified toxin.

The pharmaceutical or immunogenic compositions may be formulated in a variety of ways such as, but not limited to, solutions, suspensions, emulsions in oily or aqueous vehicles, pastes, and implantable sustained-release or biodegradable formulations. Such formulations may comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Other parenterally-administrable formulations, which are useful, include ones that comprise the active ingredient in microcrystalline form, in a liposomal preparation, or as a component of a biodegradable polymer system, e.g., a microparticles or nanoparticles. In some embodiments a sustained release formulation is used. In some embodiments, a composition is administered enterally, i.e., to any portion of the gastrointestinal tract. For example, oral administration may be used. The modified AB₅ toxin may be formulated in a way designed to reduce digestion by acid or proteolytic enzymes in the stomach or duodenum.

Additional components that may be included in the immunogenic compositions of this invention are adjuvants (in addition to the modified AB₅ toxin), preservatives, chemical stabilizers, or other antigenic proteins. Stabilizers, adjuvants, and preservatives may be optimized to determine an optimal formulation for efficacy in the target human or animal. Suitable exemplary preservatives include chlorobutanol, potassium sorbate, sorbic acid, sulfur dioxide, propyl gallate, the parabens, ethyl vanillin, glycerin, phenol, and parachlorophenol. Suitable stabilizing ingredients that may be used include, for example, casamino acids, sucrose, gelatin, phenol red, N—Z amine, monopotassium diphosphate, lactose, lactalbumin hydrolysate, and dried milk. Exemplary conventional adjuvants include, without limitation, 3-O-deacylated monophosphoryl lipid A, synthetic lipid A analogs or aminoalkyl glucosamine phosphate compounds (AGP), or derivatives or analogs thereof (see, e.g., U.S. Pat. No. 6,113,918). Other conventional adjuvants include mineral oil and water emulsions, aluminum salts (alum), such as aluminum hydroxide, aluminum phosphate, etc., Amphigen, Avridine, L121/squalene, D-lactide-polylactide/glycoside, pluronic polyols, muramyl dipeptide, killed Bordetella, saponins (U.S. Pat. No. 5,057,540), particles such as ISCOMS (immunostimulating complexes), Mycobacterium tuberculosis, bacterial lipopolysaccharides, synthetic polynucleotides such as oligonucleotides containing a CpG motif, etc. In some embodiments, adjuvants (other than the modified AB5 toxin) are not included in the composition, i.e., the composition is substantially free of such adjuvants. A composition may be considered “substantially free” of a substance if, e.g., the composition contains 1% or less, e.g., 0.1% or less, e.g., 0.05% or less, e.g., 0.01% or less, 0.005% or less, e.g., 0.001% or less, e.g., 0.0005% or less, e.g., 0.0001% or less, of a substance by weight or by moles. In some embodiments a composition is “substantially free” of a component if the component is not detectable using a standard detection method used in the art for detecting such component. In some embodiments a composition is “substantially free” of a component if the component is not deliberately added to a composition and is not expected to be present in any of the constituents used to produce the composition.

In some embodiments, an immunogenic composition of the invention contains, in addition to a modified AB₅ toxin comprising an antigen against which an immune response is desired, one or more additional AB₅ toxins or portions thereof (e.g., a B subunit), which may provide additional adjuvant effect. The additional toxin may be, e.g., PT or LT. If a portion comprising the enzymatic component is administered, a detoxified variant thereof may be used.

Additional suitable components that may be present in the immunogenic compositions of this invention include, but are not limited to: surface active substances (e.g., hexadecylamine, octadecylamine, octadecyl amino acid esters, lysolecithin, dimethyl-dioctadecylammonium bromide), methoxyhexadecylgylcerol, and pluronic polyols; polyamines, e.g., pyran, dextransulfate, poly IC, carbopol; peptides, e.g., muramyl dipeptide, dimethylglycine, tuftsin; oil emulsions; and mineral gels, e.g., aluminum phosphate, etc. and immune stimulating complexes. The modified AB₅ toxin of the invention may be incorporated into liposomes or other lipid-based particles, or conjugated to polysaccharides, lipopolysaccharides and/or other polymers for use in an immunogenic composition. In other embodiments a modified AB₅ toxin is incorporated into microparticles or nanoparticles, e.g., comprised of biocompatible, e.g., biodegradable, polymers.

An immunogenic composition of the invention may be administered to a subject in need thereof, e.g., a subject at risk of or suffering from a tumor, infection, autoimmune disease, or disease associated with a pathogenic endogenous protein. The composition can be administered prophylactically or after the subject has been infected or diagnosed with the disease. In some embodiments the subject has been identified as being at risk of the disease, e.g., at increased risk relative to many or most members of the general population. Such identification could be based at least in part on, e.g., the subject's family history, medical history, travel history, genetic analysis, appropriate clinical or laboratory diagnostic tests, etc. In some embodiments the composition is administered to treat a subject suffering from a tumor. In some embodiments the subject also undergoes or has undergone other therapy for the tumor (e.g., surgery, radiation, chemotherapy). The tumor can be any tumor, e.g., any tumor that expresses a tumor-associated antigen. In some embodiments the subject suffers from an infection with a pathogen or has been exposed to the pathogen and is at risk of infection. In some embodiments the subject is immunocompromised, e.g., the subject suffers from an an inherited or acquired immunodeficiency or is undergoing therapy with an immunosuppressive agent (e.g., to prevent rejection of a transplant). In some embodiments the subject is an infant (e.g., under 6 months of age), or under 2 years of age, or under 5 years of age. In some embodiments the inventive composition is used together with one or more conventional treatments for the particular disease. In some embodiments an inventive composition and a conventional therapeutic agent are administered in the same composition while in other embodiments they are administered separately.

In some embodiments a composition of the invention is administered to an animal that serves as a model for a disease of interest. The animal may have been exposed to a pathogen, bear an experimentally induced tumor (e.g., a tumor xenograft), have an experimentally induced autoimmune disease, etc. Such methods may be used, e.g., to evaluate efficacy and/or to study the disease.

A pharmaceutical or vaccine composition of the invention can be administered to a subject using any suitable route of administration. Suitable routes of administration include, but are not limited to, intranasal, oral, vaginal, rectal, parenteral, intradermal, transdermal, intramuscular, intraperitoneal, by inhalation, subcutaneous, intravenous and intraarterial. The appropriate route may be selected depending, e.g., on the nature of the immunogenic composition used, and optionally an evaluation, e.g., by a health care provider, of the age, weight, sex and general health of the patient and the antigen(s) present in the immunogenic composition, etc. In general, selection of the appropriate “effective amount” or dosage for the modified A1 chain or AB5 toxin comprising a modified A1 chain and/or other components of the immunogenic composition(s) of the present invention may also be based upon the particular identity of the AB5 toxin and/or antigen(s) as well as the physical condition of the subject, e.g., the general health, age, and weight of the subject. Such selection and upward or downward adjustment of the effective dose is within the skill of the art. The amount of A1 chain, AB5 toxin, and/or antigen required to induce an immune response, preferably a protective response, or produce a protective or therapeutic effect in the subject without significant adverse side effects may vary depending upon these factors. Suitable doses are readily determined by persons skilled in the art.

In some embodiments a dose of a composition comprising a modified A1 chain or AB₅ toxin protein, may comprise between about 1 μg to about 20 mg of the protein per mL of a sterile solution. In some embodiments the dose administered to a subject may be, e.g., between 1 μg to about 20 mg protein. Other dosage ranges may also be contemplated by one of skill in the art. An initial dose may optionally be followed by one or more additional doses if desired. The number of doses and the dosage regimen for the composition are also readily determined by persons skilled in the art. Protection may be conferred by a single dose of the immunogenic composition containing the modified A1 chain or AB₅ toxin comprising a modified A1 chain, or may require the administration of several doses, in addition, optionally, to one or more further doses at later times to maintain protection. Doses may be administered, e.g., several weeks, months, or years apart. The levels of immune response and/or immunity can be monitored to determine the need, if any, for additional doses.

In some embodiments, the cytoplasmic delivery and/or adjuvant propert(ies) of the modified A1 chain or AB₅ toxin may reduce the number of doses containing antigen that are needed to achieve a desired response or level of immunity. In some embodiments, administration of an inventive immunogenic composition generates a primary CD8+ T cell response against the antigen.

In some embodiments of interest a vaccine composition of the invention is administered such that it contacts a mucosal surface. For example, the composition is administered orally, vaginally, or nasally.

In some embodiments the composition is administered transcutaneously using a patch. The invention provides patch comprising an inventive modified toxin. In some embodiments the patch comprises an adhesive material useful to adhere the patch to the skin.

In some embodiments, a modified AB₅ toxin having an antigen attached thereto is used to prepare a composition for cell therapy. For example, a modified AB₅ toxin having an antigen (e.g., a tumor-associated antigen) attached to its A1 chain is contacted with cells ex vivo. The cells may be, e.g., human cells. The cells may be immunologically matched with a subject (e.g., allogeneic cells) or may be isolated from a subject (e.g., autologous cells). The subject may be suffering from a tumor or from an infection such as HIV infection. In some embodiments the antigen comprises material obtained from the tumor (e.g., peptides derived from tumor cells obtained from the subject). The cells contacted with the modified AB₅ toxin can comprise, e.g., dendritic cells, T cells (e.g., CD8+ T cells), antigen-presenting cells, NK cells, or any cells that may be of use to generate an immune response. The cells are contacted with the modified AB₅ toxin in a suitable medium in an appropriate vessel, e.g., a dish, flask, etc. In some embodiments the cells are expanded in culture prior to or while being contacted with the modified AB₅ toxin. In some embodiments the cells are also contacted with an immunomodulating agent, e.g., an immunostimulating agent (e.g., IL-2 or an interferon) while in culture. After a suitable period of time the cells are administered to the subject. In some embodiments a subpopulation of cells is isolated, e.g., based on expression of cell surface markers, e.g., so that a composition comprising cells only or primarily of a particular type (e.g., T cells), or largely or completely lacking cells of a particular type, is administered to the subject. In some embodiments the cells are administered intravenously, e.g., by IV infusion.

D. Screening Methods

Another aspect of the invention relates to using a modified engineered multi-chain or multi-subunit toxin to screen for agents that inhibit one or more biological activities of the toxin. For example, one can screen for compounds that inhibit toxin uptake by a target cell or that inhibit entry of the toxic portion of the toxin (e.g., the A1 chain of an AB₅ toxin) into the cell cytoplasm or that inhibit interaction of the toxic portion with its molecular target. As noted above, certain exotoxins are associated with a variety of diseases and unfortunately are considered potential biological warfare agents. Compounds that inhibit toxin uptake by a target cell, inhibit entry of the toxic portion of the toxin into the cytoplasm, and/or inhibit interaction of the toxic portion with its molecular target find use in treating individuals who have been exposed to the exotoxin, or that have been exposed to or infected by, a pathogen that produces the exotoxin.

In another aspect, a modified engineered multi-chain or multi-subunit toxin of the invention may be used to identify agents that modulate intracellular protein trafficking.

A variety of different screening approaches can be used. A toxin may be modified by ligating a detectable label (e.g., a fluorescent label) to the toxic moiety, thereby allowing its visualization using suitable imaging techniques such as fluorescence microscopy, or detection by flow cytometry, etc.

A wide variety of compounds may be screened. For example, candidate compounds could be proteins, peptides, nucleic acids, small organic molecules (by which is meant an organic compound less than 2 kD in molecular weight usually having multiple carbon-carbon bonds), carbohydrates, lipids, etc. In some embodiments a library comprising at least 1,000, at least 10,000, or at least 100,000 compounds is screened. In some embodiments the compounds are natural products. In some embodiments synthetic compounds are screened. One of skill in the art will be able to implement appropriate screening methods. See, e.g., WO/2008/103966 (PCT/US2008/054809) for further information regarding compounds that can be screened, screening methods, and other information that may be applied in the context of the present invention.

In another aspect, modified engineered multi-chain or multi-subunit proteins can be used to identify endogenous biomolecules, e.g., endogenous proteins, that play a role in intracellular protein trafficking. For example, a toxin may be modified by ligating a photo-activatable cross-linking agent to the toxic moiety. The toxin is contacted with eukaryotic cells. After a sufficient period of time to allow toxin uptake, the cross-linker is activated, and the toxin is cross-linked to nearby cellular biomolecules. The complex is isolated and the attached biomolecules are identified, e.g., by mass spectrometry, peptide sequencing, etc. The biomolecule is a target for identifying agents that modulate intracellular protein trafficking.

For example, a CT or LT A1 chain is labeled with a fluorophore and contacted with living cells, and the trafficking of the A1 chain is observed using a fluorescence-based imaging technique.

E. Kits

The invention further provides a variety of kits. Kits containing any of the inventive engineered polynucleotides, engineered precursor polypeptides and/or engineered multi-chain or multi-subunit proteins of the invention are contemplated. In some embodiments the kit contains an engineered precursor polypeptide of the invention. In some embodiments the kit contains an engineered precursor polypeptide in which a transamidase recognition sequence is located no more than 30 amino acids from a cleavage site. In some embodiments a kit contains an engineered multi-subunit protein of the invention, e.g., an engineered CT or LT variant in which a transamidase recognition sequence is present near the C-terminus of the A1 chain. The protein may be cleaved or uncleaved. In some embodiments the protein is modified, e.g., a compound of interest is ligated to the A1 chain. In other embodiments the protein is not modified. The user of the kit may ligate a compound of interest to the A1 chain. In some embodiments the kit comprises a nucleic acid or vector that encodes an inventive engineered precursor polypeptide, e.g., an A chain of an AB₅ toxin. In some embodiments the kit contains a nucleic acid or vector that encodes the A and B subunits of an AB5 toxin, e.g., a bicistronic vector. In some embodiments the kit further contains a nucleic acid or vector that encodes the B chain of an AB₅ toxin. In some embodiments the kit contains nucleic acids or vectors that encode the A and B subunits of an AB₁ toxin. In some embodiments the kits comprise a transamidase, e.g., sortase A. Kits may comprise any one or more of the foregoing components. A kit may also comprise, e.g., a buffer, a protease (which may be immobilized on a support), a compound of interest, and/or instructions for use of the kit, e.g., to ligate a compound of interest to a polypeptide generated by cleavage of the precursor polypeptide.

Those skilled in the art will recognize, or be able to ascertain using no more than routine experimentation, many equivalents to the specific embodiments of the invention described herein. The scope of the present invention is not intended to be limited to the above Description or the details set forth in the Examples, which are not intended to limit the invention in any way. Articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims (whether original or subsequently added claims) is introduced into another claim (whether original or subsequently added). In particular, any claim that is dependent on another claim can be modified to include one or more elements or limitations found in any other claim that is dependent on the same base claim. Furthermore, where the claims recite a composition, the invention provides methods of making the composition, e.g., according to methods disclosed herein, and methods of using the composition, e.g., for purposes disclosed herein. Also, where the claims recite a method of making a composition, the invention provides compositions made according to the inventive methods and methods of using the composition, unless otherwise indicated or unless one of ordinary skill in the art would recognize that a contradiction or inconsistency would arise.

Where elements are presented as lists, e.g., in Markush group format, each subgroup of the elements is also disclosed, and any element(s) can be removed from the group. For purposes of conciseness only some of these embodiments have been specifically recited herein, but the invention includes all such embodiments. It should also be understood that, in general, where the invention, or aspects of the invention, is/are referred to as comprising particular elements, features, etc., certain embodiments of the invention or aspects of the invention consist, or consist essentially of, such elements, features, etc.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where phrases such as “less than X”, “greater than X”, or “at least X” is used (where X is a number or percentage), it should be understood that any reasonable value can be selected as the lower or upper limit of the range. It is also understood that where a list of numerical values is stated herein (whether or not prefaced by “at least”), the invention includes embodiments that relate analogously to any intervening value or range defined by any two values in the list, and that the lowest value may be taken as a minimum and the greatest value may be taken as a maximum. Furthermore, where a list of numbers, e.g., percentages, is prefaced by “at least”, the term applies to each number in the list. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments 5% or in some embodiments 10% of a number in either direction (greater than or less than the number) unless otherwise stated or otherwise evident from the context (e.g., where such number would impermissibly exceed 100% of a possible value).

In addition, any particular embodiment(s), aspect(s), element(s), feature(s), etc., of the present invention, e.g., any precursor polypeptide, multi-chain or multi-subunit protein, compound of interest, may be explicitly excluded from the claims.

### EXEMPLIFICATION

### Example 1

### Efficient Labeling of Cholera Toxin A1 Chain Using Sortase

### Materials and Methods

Expression and Purification of Modified Cholera Holotoxin.

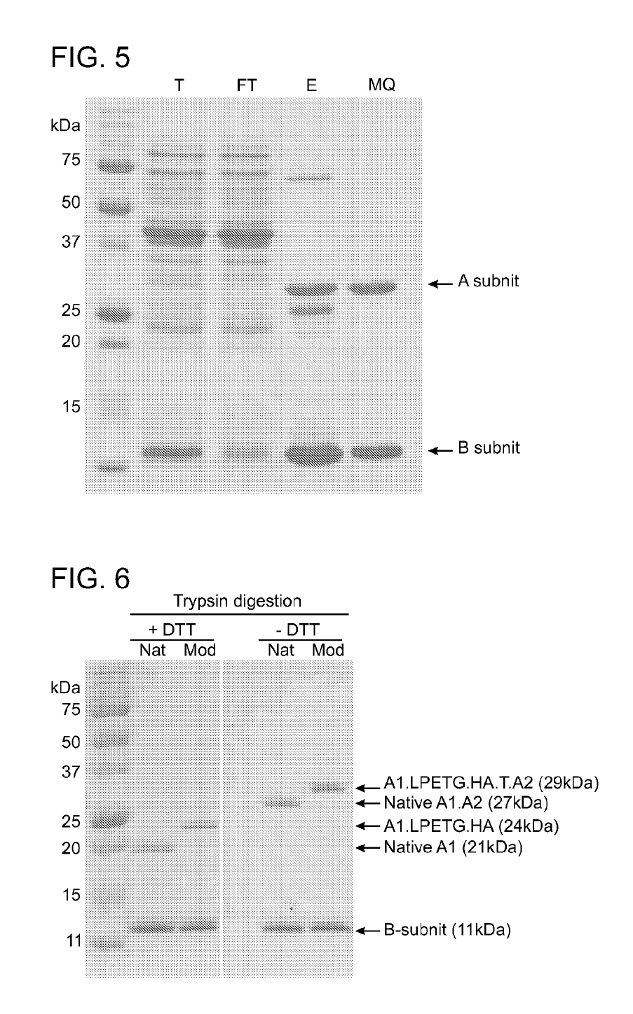

Sterile Luria-broth media containing antibiotic (chloramphenicol 35 μg/ml) is inoculated with a single colony of BL21 harboring the plasmid encoding for the sortaggable loop version of cholera toxin (FIG. 4c). The culture is grown for 16 hours at 30° C. with vigorous shaking. This pre-culture is then diluted (1:50) in Terrific Broth media (prepared fresh and not autoclaved) plus antibiotic. The culture is grown at 37° C. with agitation. When the bacterial density reaches an optical density of 0.6 at A600 nm (approximately after 2 hours), expression of cholera toxin is induced by addition of arabinose 0.25% (w/w) plus antibiotic, for 4 hours at 37° C. The cells are then harvested by centrifugation and frozen at −20° C. Since cholera toxin is expressed in the periplasm, the first step of the purification protocol is to disrupt the cell wall releasing all the periplasmic proteins. For this, each bacterial cell pellet, derived from 1 L of culture, is gently resuspended in buffer A (20 ml of 20 mM Tris-Cl pH 8.0, 0.3M NaCl) supplemented with 1 mg/ml polymixin B sulfate and with an EDTA-free protease inhibitor cocktail. Incubation on an end-over-end shaker occurs for 1 hr at 25° C. The spheroplasts are then removed by centrifugation and the corresponding supernatant (FIG. 5, lane T) is incubated with Ni-NTA beads (Qiagen), at 4° C. for 30 minutes. The beads are then poured onto disposable columns and extensively washed with cold buffer A. Proteins are eluted using 20 mM Tris-Cl pH 8.0, 0.15M NaCl, 0.3M imidazole (FIG. 5, lane E). The eluate is then diluted 10 times with 20 mM Tris-Cl, pH 8.0 and further purified by high-resolution anion exchange chromatography (Mono Q). The proteins are eluted from the column with a linear salt gradient. The fractions containing the holotoxin are pooled (FIG. 5, lane MQ) and the protein concentration is determined. These preparations of cholera toxin are very stable and can be stored for several months at 4° C.

Results

We sought to apply the sortagging strategy to specifically label the A1 chain of cholera toxin. Sortagging was selected since it is able to install a variety of molecules, in a specific manner, onto a protein. Also, sortase A is able to act on proteins that are already folded. Since cholera toxin is a heteromer, we reasoned that if the labeling of one of the subunits had to be done separately, then the hexameric structure complex would have to be restored. Using a pre-formed complex avoids technical problems inherent to any in vitro reconstitution. In addition, having a large preparation of unlabeled toxin ready to be labeled is convenient and helps ensure experimental reproducibility.

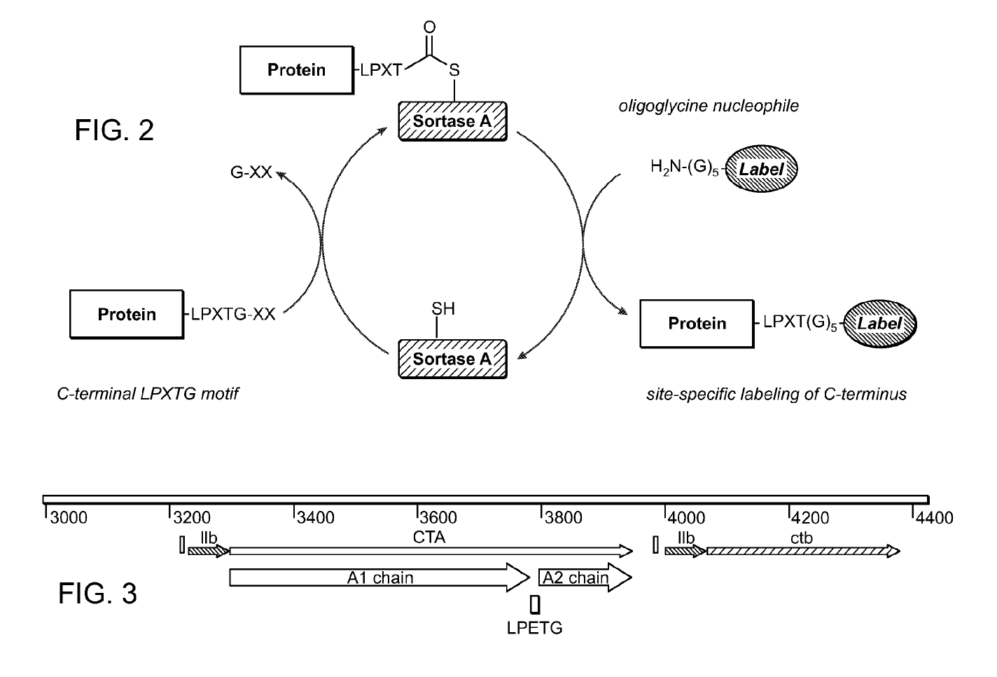

We selected the A1 region as a target for labeling in part because it contains the enzymatic toxic portion, which is only active when it reaches the cytosol. Therefore, the ability to place a probe in this sequence could serve multiple purposes (as discussed elsewhere herein). We recognized that one of the requisites for an efficient sortase-catalyzed transpeptidation reaction to occur is the installation of the recognition motif into a flexible and accessible region of the protein. Given this, the LPXTG sequence is usually cloned at the C-terminus of the substrate protein (as represented in FIG. 2). We examined the three-dimensional crystal structure of the cholera holotoxin and observed that the region that contains the protease sensitive loop between the A1 and A2 portions of the A chain is disordered in the structure, which suggested that it is a flexible region. We reasoned that cleavage of the loop, mimicking what happens in nature, would facilitate covalent attachment of a molecule of choice to the downstream part of the A1 chain by sortase while minimizing the likelihood of disrupting the ability of the A1 chain to translocate to the cytosol.

Data indicate that serine endoproteases, which are abundant both in bacteria and mammalian cells, are able to efficiently cleave the protease sensitive loop of cholera toxin, at position Arginine 192. Since cholera toxin was to be expressed in bacteria, we wanted to make sure that in the labeling strategy cleavage of the loop would occur solely by the action of sortase A. Therefore, we replaced the amino acids Proline 191 and Arginine 192 in the A subunit sequence by a LPETG motif, which is recognized by sortase A. For expression, a bicistronic bacterial vector coding for the recombinant A subunit (p.Pro191_Arg192delinsLeuProlGluThrGly) followed downstream by one native B subunit was used (FIG. 3). The template vector used to create the sortaggable form of cholera toxin was derived from the pAR3 vector and is arabinose inducible (Pérez-Pérez J and Gutierrez J (1995) Gene 158:141-142). Both A and B subunits of cholera toxin are synthesized as precursor proteins, with signal sequences from the B subunit of the Escherichia coli heat-labile enterotoxin LTII-b. Both subunits are synthesized as precursor proteins, with signal sequences from the B subunit of the Escherichia coli heat-labile enterotoxin LTII-b. Due to these sequences, the precursor proteins are transported to the periplasm, where they are processed and associate to form the holotoxin (i.e., A subunit in association with the B ring) (Jobling M G et al (1997) Plasmid 38:158-173; Hardy S J S et al (1988) PNAS 85:7109-7113).

The next step was to test whether this modified version of cholera toxin was in fact substrate for sortase-mediated transpeptidation. In the initial experiments general labeling conditions that had already been described for other proteins were used (Popp M W et al (2007) Nat Chem Biol 3:707-708). The preliminary results indicated that a fraction of cholera toxin was being labeled by sortase A. However, the efficiency of labeling was relatively low, and we therefore sought means to improve it. We reasoned that, although the loop region seems to be structurally flexible (as the crystal structure suggests), it must still impose some constrains for the action of sortase. In an attempt to overcome this potential limitation, the size of the loop was increased. However, no improvement was observed. Considering that sortase reactions using protein substrates containing the LPXTG motif positioned at their C-terminus are usually very efficient, we decided to test whether opening the loop prior to the action of sortase would increase the labeling reaction efficiency. We reasoned that cleaving the loop first with a protease would release some of the constraints imposed by a closed loop, since now the end recognized by sortase A would have some structural freedom. To test this idea, a nucleic acid construct was generated that encodes a modified version of the cholera toxin A chain in which a sequence that is recognized by the serine endoprotease trypsin was positioned downstream of the LPETG motif (compare FIGS. 4a, 4b and 4c). In addition, the modified version of the A chain contains an HA tag (YPYDVPDYA) positioned between the LPETG motif and the trypsin cleavage site. The presence of this epitope in this position allows the efficiency of the sortagging reaction to be determined by immunoblotting analysis, since the HA sequence is cleaved off upon sortase-mediated transpeptidation. The sequence of the resulting engineered A subunit is as follows: NDDKLYRADSRPPDEIKQSGGLMPRGQSEYFDRGTQMNINLYDHARGTQTGFVRHD DGYVSTSISLRSAHLVGQTILSGHSTYYIYVIATAPNMFNVNDVLGAYSPHPDEQEVS ALGGIPYSQIYGWYRVHFGVLDEQLHRNRGYRDRYYSNLDIAPAADGYGLAGFPPE HRAWREEPWIHHAPPGCGNALPETGGYPYDVPDYAMNAPRSSMSNTCDEKTQSLG VKFLDEYQSKVKRQIFSGYQSDIDTHNRIKDEL. The additional amino acids, relative to the wild type sequence, are underlined. As noted above, two residues (Pro191 and Arg192) were deleted and a segment with sequence LPETGGYPYDVPDYAMNAPR was inserted. Since this sequence has Pro and Arg at its C-terminus the net effect was the addition of LPETGGYPYDVPDYAMNA between Ala190 and Pro191 in the native sequence. In summary, the segment between the two cysteine residues consisted of GNA (from the A1 chain), LPETG (sortase recognition motif), G downstream of sortase recognition motif, HA tag (YPYDVPDYA), M (serving as an amino acid spacer but not required), NAPR (same sequence as amino acids 189-193 of the native A1 chain). Trypsin recognizes PR and cleaves after the arginine.

The E. coli BL21 strain and a rich media (Terrific Broth) were used for expression and the holotoxin was purified after disruption of the bacterial outer membrane. Steps in the purification are shown in FIG. 5. Purification of cholera toxin. Lane T—Periplasmic proteins released upon disruption of the outer membrane with polymixin B. Lane FT—Flow-through upon binding to Ni-NTA beads. Lane E—Eluate from the beads. Lane MQ—Pooled eluate fractions containing holotoxin, upon purification through a Mono Q column. The samples were analyzed onto a 12% SDS-PAGE under reducing conditions. The gel was stained with Coomassie blue. The molecular standards are shown in kDa. The two subunits of cholera toxin are indicated by arrows. The spheroplasts are then removed by centrifugation and the corresponding supernatant (FIG. 5, lane T) is incubated with Ni-NTA beads (Qiagen), at 4° C. for 30 minutes. The beads are then poured onto disposable columns and extensively washed with cold buffer A. Proteins are eluted using 20 mM Tris-Cl pH 8.0, 0.15M NaCl, 0.3M imidazole (FIG. 5, lane E). The eluate is then diluted 10 times with 20 mM Tris-Cl, pH 8.0 and further purified by high-resolution anion exchange chromatography (Mono Q). The proteins are eluted from the column with a linear salt gradient. The fractions containing the holotoxin are pooled (FIG. 5, lane MQ) and the protein concentration is determined. We were able to express batches with typical yields of approximately 0.8-1.2 mg of pure holotoxin per liter of culture.

The subsequent step after purification of cholera toxin is cleavage of the engineered loop by trypsin (EC 3.4.21.4). Trypsin is a serine protease that cleaves mostly peptide chains at the carboxyl side of the amino acids lysine and arginine, except (usually) when these residues are followed by a proline residue. To avoid an extra purification step after trypsin digestion, we used TPCK immobilized trypsin (Pierce #20230) in our protocol, allowing us to efficiently remove trypsin from the preparations. Removal of trypsin was desired in order to avoid digestion of sortase A during the transpeptidation assays. We use 5 μl of a 50% slurry for each 1 mg of cholera toxin. The incubation is performed at room temperature, in an end-over-end shaker, for 90 minutes. After this time an aliquot is analyzed by reducing SDS-PAGE to confirm the extent of cleavage. After cleavage the sample is centrifuged through a 0.22 μm nylon membrane filter tube (Costar #8169), at 9000×g for 2 min, to efficiently remove the trypsin-immobilized beads from our preparations.

An important consideration when using extended loop versions in the context of cholera toxin is that the disulfide bridge, which holds A1 and A2 chains together (FIG. 1), has to form and stay intact during the whole purification procedure and after cleavage of the loop. To assess this, we analyzed the product resultant from digestion of the purified cholera toxin with trypsin by SDS-PAGE, under reducing and non-reducing conditions. As shown in FIG. 6, the 29 kDa protein band (corresponding to the A subunit containing the LPETG motif, as depicted in FIG. 4c), upon incubation with trypsin, shifts to the region of the 24 kDa, only under reducing conditions (+DTT). This result indicates that upon nicking the loop with trypsin and reducing the disulfide bridge with DTT, the A1 and A2 chains separate and migrate according to their individual molecular weights (A1 chain=24 kDa/A2 chain=5.5 kDa). However, in the absence of DTT the nicked A subunit containing the LPETG motif migrates as a 29 kDa protein, showing that the A1 and A2 chains remain bound by the disulfide bridge. The same behavior is observed for the native cholera toxin construct (i.e., native loop). Therefore, we concluded that our engineered loop maintains the features of the native structure of cholera toxin.

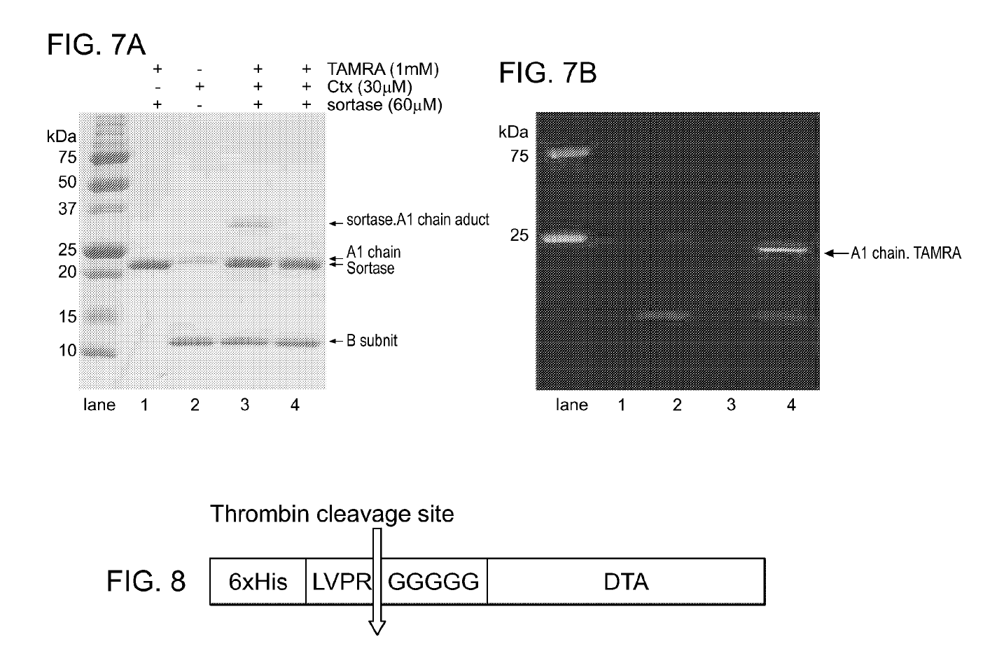

As we had hypothesized, cleavage of the loop with trypsin before the sortase coupling reaction increases the efficiency of labeling. As can be observed in FIG. 7, the protein band corresponding to the A1 chain subunit (lane 2, 7A) is labeled only when sortase A, cholera toxin and nucleophile (in this case the fluorophore TAMRA) are incubated together (lane 4). We have been successful in decorating the A1 chain of cholera toxin with all the labels tested so far, such as biotin, small peptides (8 mer), and large proteins (ca. 20 kDa, such as GFP and the catalytic chain of diphtheria toxin).

### Example 2

### Use of the A1 Chain of Cholera Toxin to Deliver Proteins to the Cytosol of Mammalian Cells

One of the proteins that we have conjugated to the A1 chain of cholera toxin is the catalytic site of diphtheria toxin. Diphtheria toxin is composed of two subunits: DTA (diphtheria toxin subunit A), which is the toxic part, and DTB (diphtheria toxin subunit B), which binds to the cellular receptor and allows DTA to enter the cell. The substrate for diphtheria toxin is diphthamide, a modified histidine amino acid in the eukaryotic elongation factor 2 (eEF-2). Diphtheria toxin renders this elongation factor inactive by ADP-ribosylation, resulting in impairment of protein synthesis, leading to cell death (Deng, Q. & Barbieri, J. T. (2008) Annu Rev Microbiol 62, 271-88.). To be active, DTA needs to reach the cytosol where its substrate resides. DTA is a protein of approximately 20 kDa (194 amino acids). Considering that this protein by itself is unable to bind to the plasma membrane and therefore to intoxicate cells, we asked whether the A1 chain of cholera toxin could transport and deliver a protein of about its size to the cytosol. If that was the case, the read out would be cell death, due to the action of DTA.

To be able to use DTA as a nucleophile in a sortase-mediated reaction, we needed to clone a pentaglycine extension at the N-terminus of the protein (as schematized in FIG. 2). For this, we made use of the vector pET-15b LFN-DTA (Addgene). This plasmid contains the sequences for both the N-terminal domain of the anthrax lethal factor (LFN) and DTA. Therefore, we replaced the entire LFN sequence by a pentaglycine coding region. The final version of the construct contains a 6×His tag that allows purification of the protein (using a Ni-NTA column), followed by a thrombin cleavage site that allows removal of the 6×His tag and exposure of the 5 glycines, which precede the catalytic active site of DTA (FIG. 8). Expression of the construct was done in BL21(DE3) E. coli strain for maximal expression using Luria-broth media. Upon purification, the protein was incubated with immobilized thrombin (which cleaves between the arginine and glycine residues as indicated in FIG. 8), leading to the final version of the protein: GGGGG-DTA.

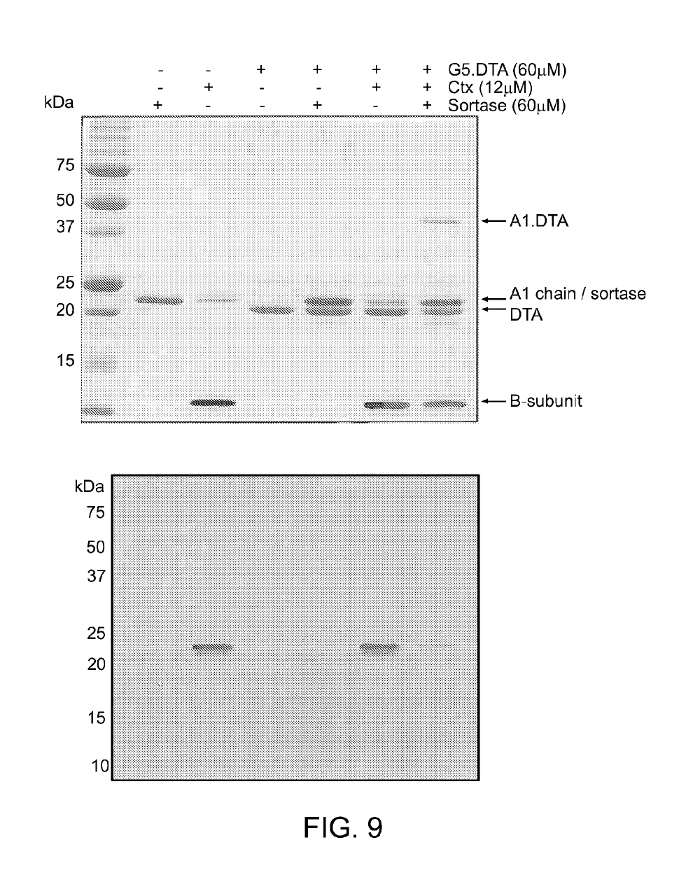

Using a purified sortaggable cholera holotoxin (as described in Example 1), with the loop nicked by trypsin (as described in FIG. 4c), we tested the efficacy of sortase A to mediate the ligation between GGGGG-DTA and the A1 chain of cholera toxin. The results are shown in FIG. 9. As can be observed in FIG. 9 (upper panel), a new protein band of approximately 40 kDa appears only in the reaction tube that contains the following components: sortase A, plus cholera toxin, plus DTA. This protein band was excised from the gel and its identity was determined by mass-spectrometry confirming that it is in fact the A1 chain coupled to DTA (data not shown). The efficiency of the reaction was assessed by immunoblotting using an antibody directed to the HA epitope (FIG. 9, lower panel). As shown in FIG. 4c, the HA tag that is cloned downstream the sortase recognition motif is removed upon sortase-mediate transpeptidation. In this case, the levels of HA detected upon sortase reaction are very low compared to the input levels, suggesting that the reaction took place with high efficiency. Examination of the Coomassie-stained gel confirmed stoichiometric conversion of A1 to A1-DTA.

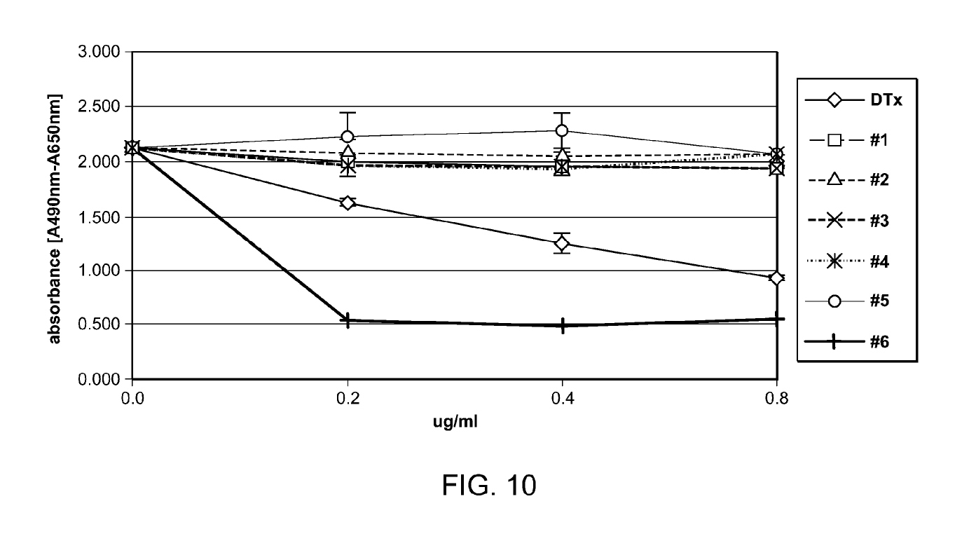

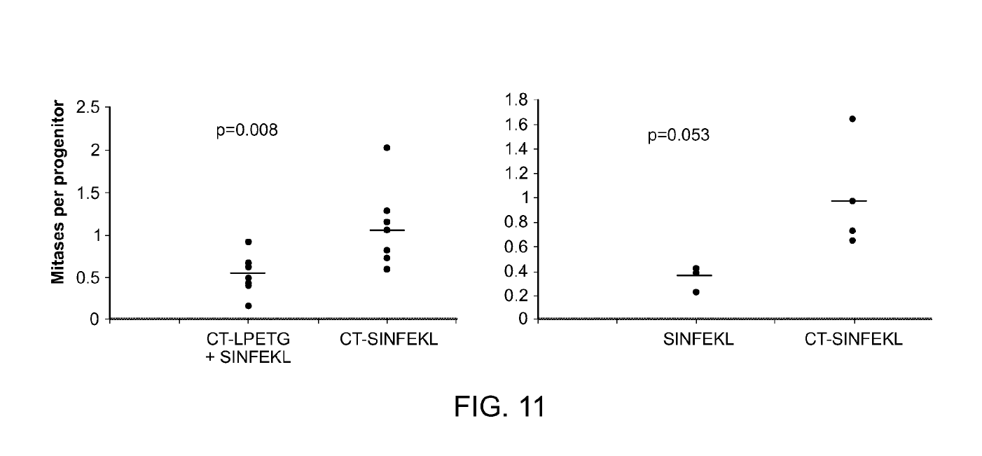

The next step was to test whether this DTA-labeled version of cholera toxin was lethal to cells. If so, this would mean that DTA had been delivered to the cytosol and had interacted with its substrate. To address this, we plated the same number of cells on each well of a 96-well plate and intoxicated the cells with different volume amounts taken from the reactions shown in FIG. 9. The cells were incubated for 16 hrs, at 37° C. in a 5% CO₂ atmosphere. The cellular viability was then tested using the cytotoxic XTT assay (Roche). As shown in FIG. 10, cellular death is detected only when the cells are intoxicated with an aliquot of the reaction containing sortase A, plus DTA and cholera toxin. The efficacy of this mixture is very similar to the one observed by the chimera LFN. DTA (Addgene). In this assay, we used human KBM-7 cells but other cells (e.g., 293T cells) can also be intoxicated in the same manner (data not shown). These results indicate that DTA is reaching its substrate in the cytosol only when it is coupled to the A1 chain of cholera toxin. Also, it shows that the presence of the A1 chain does not interfere with the function of DTA. These results provide evidence that cholera toxin can be used as an effective delivery vehicle of proteins or other cargoes of interest to the cytosol when these moieties are appended to the A1 chain. To our knowledge, this represents the first reported example of a successful execution of this type of protein surgery.

### Example 3

### Sortagging the A1 Chain of an AB5 Toxin for the Development of a New Vaccine Approach

The results obtained for cholera toxin. DTA strongly suggest that polypeptide cargos (at least those containing less than 200 amino acids) are able to be transported to the cytosol of cells, when the cargo (in this example DTA) is covalently attached by sortase to the A1 chain. Based on this result we will use this method to develop a new vaccine adjuvant vector. It has been described that cholera toxin (in particular the B subunit) has strong adjuvant properties. Therefore, if it would be possible to use cholera toxin to target a cargo (polypeptide, sugar, lipid, etc), to which we want to develop an immune response, we predict that we would generate a strong new vaccine adjuvant vector. Cholera toxin has in fact been tested in this regard. However, these studies used genetically engineered recombinant cholera toxin, either fusing the polypeptide to one subunit B or to the A2 chain of cholera toxin. Nevertheless, it is the A1 chain that traffics to the cytosol and that has potential to deliver the peptides to be loaded onto MHC Class I for presentation. Therefore, we hypothesized that attaching a cargo to the A1 chain would offer certain advantages.

We explored this idea by conjugating the peptide GGGGGSIINFEKL to the A1 chain of cholera toxin using sortase. OVA257-264 (SIINFEKL) has been described as a very immunogenic peptide from the ovalbumin sequence, and tools are available to determine the effect of cholera toxin conjugated to SIINFEKL on the proliferation of OT-I T cells (which express a transgenic TCR that is specific for SIINFEKL peptide bound to H-2 Kb) that are specifically activated after intoxication of mice. We injected mice with cholera toxin (2 picomoles) that had been covalently ligated to SIINFEKL by sortase. As a control, we injected the same amount of cholera toxin and peptide (not coupled). To better compare the responses and avoid individual variability we injected the two samples in the footpads of the same mouse. After two days of intoxication the corresponding lymph nodes were extracted, and the proliferation of OT-I cells was measured. The preliminary data indicated that there is activation of these cells in the lymph node correspondent to the footpad, in which cholera toxin conjugated to SIINFEKL was injected (FIG. 10). In these assays, we used a detoxified version of cholera toxin (p. E110D, E112D), so the animal does not get sick from cholera (Jobling M G et al (2001) J Bacteriology 183:4024-4032).

We will use ovalbumin and SIINFEKL to better characterize the immune response developed upon intoxication by cholera coupled to the peptide. It will be interesting, for example, to analyze if the animals get mucosal immunity if cholera toxin peptide is administered in the nose, vagina or in the gastro-intestinal tract.

### Example 4

### Sortagging the A1 Chain of an AB5 Toxin for the Development of a New HPV Vaccine

Studies using the E6 and E7 polypeptides, from the human papilloma virus (HPV), will be performed aiming at the development and characterization of a vaccine using detoxified cholera toxin coupled to those cargos. E6 interacts with the cellular E6 associated-protein (E6AP), a HECT domain ubiquitin ligase leading to ubiquitination and degradation of the anti-tumor suppressor protein p53 (Talis, A. L., Huibregtse, J. M. & Howley, P. M. (1998) J Biol Chem 273, 6439-45). Thanks to the recently approved HPV vaccine, cervical cancer should now in theory be largely preventable, at least for the predominant serotypes covered by the approved vaccines (Group, F. I. S. (2007) N Engl J Med 356, 1915-27.). However, these HPV vaccines are just prophylactic. The ability to stimulate the immune system to eradicate already transformed cells presents an enticing possibility to achieve a therapeutic effect. Immune-mediated tumor rejection often relies at least in part on the generation of CD8+ cytotoxic T cells that recognize tumor-specific antigenic peptides presented on Class I MHC products (MHCI). Unlike Class II MHC products, which present peptides from endocytosed material degraded in the endolysosomal system, MHCI presents peptides mostly from intracellular proteins. Peptides derived from a variety of proteins can elicit protective immune responses against cancers (Brichard, V. G. & Lejeune, D. (2007) Vaccine 25 Suppl 2, B61-71; Odunsi, K., Qian, F., Matsuzaki, J., Mhawech-Fauceglia, P., Andrews, C., Hoffman, E. W., Pan, L., Ritter, G., Villella, J., Thomas, B., Rodabaugh, K., Lele, S., Shrikant, P., Old, L. J. & Gnjatic, S. (2007) Proc Natl Acad Sci USA 104, 12837-42; Kawakami, Y., Eliyahu, S., Jennings, C., Sakaguchi, K., Kang, X., Southwood, S., Robbins, P. F., Sette, A., Appella, E. & Rosenberg, S. A. (1995) J Immunol 154, 3961-8; Schmollinger, J. C., Vonderheide, R. H., Hoar, K. M., Maecker, B., Schultze, J. L., Hodi, F. S., Soiffer, R. J., Jung, K., Kuroda, M. J., Letvin, N. L., Greenfield, E. A., Mihm, M., Kutok, J. L. & Dranoff, G. (2003) Proc Natl Acad Sci USA 100, 3398-403.).

Many of these tumor rejection antigens appear to be conserved in certain types of tumors, providing attractive targets for therapeutic vaccination. However, recombinant proteins do not usually elicit CD8+ T cell responses, because the exogenously added proteins fail to enter the Class I MHC processing and presentation pathway. Instead, self-replicating vectors or other genetic means of introducing the antigen are used, with varying degrees of success and with the marked drawback of genetic alterations in the cells or tissues targeted. A strategy that relies on the simple production of a suitable protein preparation would be highly desirable.

Studies using the E6 and E7 polypeptides, from the human papilloma virus (HPV), will also be designed aiming at the development and characterization of a vaccine using detoxified cholera toxin coupled to those cargos. These are attractive candidate tumor rejection antigens given that they are constitutively expressed in HPV-transformed cells and are required for the development of cervical cancer. We will undertake sortase-mediated fusion of E6 and E7 oncoproteins to the CTA I chain. To this end, we will clone HPV16 E6 and HPV16 E7 in bacterial expression vectors in a form suitable for use in a sortase-mediated chemoenzymatic reaction (sortagging). Both the catalytically active and inactive forms of E6 and E7 will be expressed, purified and coupled to the A1 chain to obtain CTx-E6 or CTx-E7 holotoxins. Since the E6 and E7 proteins are smaller than DTA, we expect to obtain a comparable or even higher coupling yields. We will use both toxic and detoxified versions of CTx.

We will evaluate the capacity of CTA1 to correctly deliver E6 (or E7) to the cytosol. Since the E6 protein targets p53 for ubiquitin-dependent proteolysis, we will analyze the fate of p53 upon intoxication of cells in culture with the CTx-E6 sortase-mediated fusions. In a similar manner, we will assess E7 functionality analyzing the half-life of the tumor-suppressor retinoblastoma protein (pRb). These experiments should allow us to assess how effectively CTA1-E6 (or E7) molecules reach the cytosol. In parallel, we will explore the heat-labile enterotoxin from E. coli (LT) and compare the efficiency of these two toxins to deliver their cargos. The quaternary structure and mode of intoxication of LT is very similar to CTx (Dallas, W. S. & Falkow, S. (1980) Nature 288, 499-501) and therefore the fusion of antigenic proteins using sortase will be performed as described above. The use of LT has the significant advantage that its use in humans as a vaccine adjuvant has already been approved for a genetically detoxified derivative, LKT63.

We will assess activation and proliferation of E6 (or E7)-specific CD8⁺ T-cells upon intoxication with CTx and/or LT modified with cargo. Purified CTx-E6, CTx-E7 and/or LT-E6, LT-E7 fusion proteins, as well as the individual proteins, will be administered by intravaginal and intranasal routes and in the footpads of naïve mice. The immunodominant MHCI epitopes for E6 and E7 in H-2^{b} haplotype mice have been previously defined (E6⁴⁸⁻⁵⁷ EVYDFAFRDL; E7⁴⁹⁻⁵⁷ RAHYNIVTF). We have ample experience with production of H-2K^{b} and H-2D^{b} tetramers. We will use tetramer staining to quantify the number of E6 and E7 reactive CD8+ T cells that arise in mice immunized with recombinant E6/E7 versus CTx-E6 and LT-E6. Mice immunized with recombinant proteins generally do not mount CD8+ T cell responses and such animals will serve as controls. Any E6-specific CD8+ T cells generated presumably derive from successful delivery of the antigenic oncoproteins by the toxin. As an additional control, E6 and E7 specific antibody titers will be measured to assess the extent of B cell response and CD4+ helper T cell responses generated.

### Example 5

### Sortagging the A1 Chain of an AB5 Toxin for the Development of a New Influenza Virus Vaccine

Following the approach described above, we will apply the same strategy using peptides derived from the influenza virus.

1. An engineered precursor polypeptide, wherein said engineered precursor polypeptide
comprises a polypeptide of formula 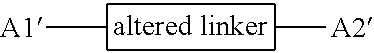
and is a variant of a naturally occurring precursor polypeptide of formula 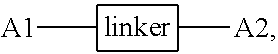
wherein: [symbol]
represents a peptide bond or polypeptide domain that comprises a first cleavage site that is cleaved during maturation of the naturally occurring precursor polypeptide;
A1′ comprises a polypeptide at least 70% identical to A1 over a substantial portion of the length of A1;
A2′ comprises a polypeptide at least 70% identical to A2 over a substantial portion of the length of A2; and [symbol]
comprises a transamidase recognition sequence and a second cleavage site.

2. The engineered precursor polypeptide of claim 1, wherein the naturally occurring precursor polypeptide is a precursor of an exotoxin or subunit thereof.

3. The engineered precursor polypeptide of claim 2, wherein the exotoxin is a bacterial exotoxin.

4. The engineered precursor polypeptide of claim 3, wherein the bacterial exotoxin is an AB₅ toxin.

5. The engineered precursor polypeptide of claim 4, wherein the naturally occurring precursor polypeptide is the A chain of the bacterial exotoxin.

6-29. (canceled)

30. A method of producing an engineered mature polypeptide comprising steps of:
(a) providing an engineered precursor polypeptide according to claim 1; and
(b) contacting the engineered precursor polypeptide with a protease that cleaves the second protease cleavage site under conditions suitable for cleavage to occur, thereby producing an engineered mature polypeptide.

31-32. (canceled)

33. A method of generating a modified, engineered mature protein comprising the step of:
(a) providing an engineered precursor polypeptide of claim 1;
(b) contacting the engineered precursor polypeptide with a protease that cleaves the second protease cleavage site under conditions suitable for cleavage to occur, thereby producing an engineered mature protein;
(c) contacting the engineered mature polypeptide with a compound that comprises an NH₂—CH₂— moiety in the presence of a transamidase, wherein the transamidase ligates the compound to the engineered mature protein, thereby generating a modified, engineered mature protein.

34. The method of claim 33, wherein the compound has formula (G)ₖ-Z¹; wherein
Z¹ is or comprises acyl, substituted or unsubstituted aliphatic, substituted or unsubstituted heteroaliphatic, substituted or unsubstituted aryl, substituted or unsubstituted heteroaryl, a peptide, a protein, a polynucleotide, a sugar, a tag, a metal atom, a contrast agent, a catalyst, a non-polypeptide polymer, a specific binding pair member, a cross-linkable moiety, a small molecule, a lipid, a photoaffinity probe, a particle, or a label; and
k is an integer from 1 to 6, inclusive.

35. (canceled)

36. The method of claim 33, wherein the compound comprises an antigen of interest.

37-62. (canceled)

63. An engineered multi-subunit precursor protein that comprises the engineered precursor polypeptide of claim 1 and a non-covalently associated protein subunit of formula (B′)ₙ, wherein n is between 1 and 6, and wherein each B′ is independently at least 70% identical to a subunit B of a naturally occurring multi-subunit protein A(B′)ₙ over a substantial portion of the length of B, and wherein A represents

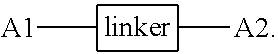

64-85. (canceled)

86. A modified engineered polypeptide of formula: 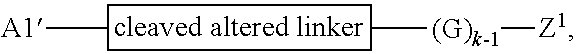
wherein [symbol]
is a polypeptide that comprises a transamidase recognition sequence;
A1′ is at least 70% identical to a polypeptide chain A1 of a naturally occurring multi-subunit protein;
k is between 0 and 6; and
Z¹ is or comprises acyl, substituted or unsubstituted aliphatic, substituted or unsubstituted heteroaliphatic, substituted or unsubstituted aryl, substituted or unsubstituted heteroaryl, a peptide, a protein, a polynucleotide, a sugar, a tag, a metal atom, a contrast agent, a catalyst, a non-polypeptide polymer, a specific binding pair member, a cross-linkable moiety, a small molecule, a lipid, a photoaffinity probe, a particle, or a label.

87. A modified engineered multi-subunit protein comprising the modified engineered polypeptide of claim 86 and a noncovalently associated protein subunit of formula (B1′)ₙ, wherein n is between 1 and 6; and each B′ is independently at least 70% identical to a subunit B of the naturally occurring multi-subunit protein over a substantial portion of the length of B.

88. The modified engineered multi-subunit protein of claim 87, wherein the naturally occurring multi-subunit protein is an AB₅ toxin.

89. (canceled)

90. The modified engineered multi-subunit protein of claim 87, wherein Z¹ comprises an antigen of interest.

91-98. (canceled)

99. A modified engineered multi-chain protein of formula: 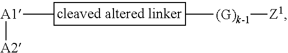
wherein [symbol]
is a polypeptide that comprises a transamidase recognition sequence;
wherein A1′ is at least 70% identical to a polypeptide A1 over a substantial portion of the length of A1,
wherein A2′ is at least 70% identical to polypeptide A2 over a substantial portion of the length of A2;
wherein A1 and A2 are naturally occurring polypeptides generated by proteolytic cleavage of a naturally occurring precursor polypeptide A1-L-A2, wherein L is an optionally present polypeptide linking domain;
wherein [symbol] comprises, in an N- to C-direction, a transamidase recognition sequence, an optionally present polypeptide spacer between 1 and 20 amino acids long, and a portion of a protease cleavage site;
k is between 1 and 6; and
Z¹ is or comprises acyl, substituted or unsubstituted aliphatic, substituted or unsubstituted heteroaliphatic, substituted or unsubstituted aryl, substituted or unsubstituted heteroaryl, a peptide, a protein, a polynucleotide, a sugar, a tag, a metal atom, a contrast agent, a catalyst, a non-polypeptide polymer, a specific binding pair member, a cross-linkable moiety, a small molecule, a lipid, a photoaffinity probe, a particle, or a label.

100. A modified engineered multi-subunit protein comprising the modified engineered multi-chain protein of claim 99 and a noncovalently associated protein subunit of formula (B1′)ₙ, wherein n is between 1 and 6; and each B′ is independently at least 70% identical to a subunit B of the naturally occurring multi-subunit protein over a substantial portion of the length of B.

101-102. (canceled)

103. The modified engineered multi-subunit protein of claim 100, wherein Z¹ comprises an antigen of interest.

104-107. (canceled)

108. A modified AB₅ toxin protein, wherein the modified AB₅ toxin protein comprises
(a) a first polypeptide chain at least 90% identical to the A1 chain of a naturally occurring AB₅ exotoxin and having a compound of interest attached thereto;
(b) a second polypeptide chain attached to the first polypeptide via a disulfide bond, wherein the second polypeptide chain is at least 90% identical to the A2 chain of the naturally occurring AB₅ exotoxin; and
(c) five additional polypeptide chains that form a subunit that is noncovalently associated with at least the second polypeptide chain, wherein each of the five additional polypeptide chains is at least 90% identical to the B chain of the naturally occurring AB₅ exotoxin.

109. The modified AB₅ toxin protein of claim 108 wherein the first polypeptide chain has formula: 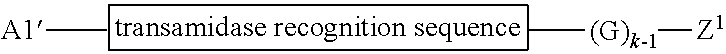
wherein A1′ is at least 70% identical to an A1 chain of a naturally occurring AB₅ toxin, n is between 0 and 6, and Z¹ is or comprises acyl, substituted or unsubstituted aliphatic, substituted or unsubstituted heteroaliphatic, substituted or unsubstituted aryl, substituted or unsubstituted heteroaryl, a peptide, a protein, a polynucleotide, a sugar, a tag, a metal atom, a contrast agent, a catalyst, a non-polypeptide polymer, a specific binding pair member, a cross-linkable moiety, a small molecule, a lipid, a photoaffinity probe, or a label; and k is an integer between 1 and 6.

110-111. (canceled)

112. The modified AB₅ toxin protein of claim 108, wherein Z¹ comprises an antigen of interest.

113-115. (canceled)

116. A method of delivering an agent of interest to the cytoplasm of a eukaryotic cell comprising contacting the cell with the modified AB₅ toxin protein of claim 108, wherein the eukaryotic cell expresses a receptor for the AB₅ toxin protein.

117. A method of delivering a compound of interest to the cytoplasm of a eukaryotic cell, the method comprising contacting the cell with a modified AB₅ toxin protein, wherein the compound of interest is linked to the A1 chain of the modified AB₅ toxin protein, and wherein the cell expresses a receptor for the AB₅ toxin protein.

118-120. (canceled)

121. The method of claim 117, wherein the compound of interest comprises an antigen of interest.

122-126. (canceled)

127. A method of generating an immune response in a subject comprising administering a modified AB₅ toxin protein to the subject, wherein the A1 chain of the modified AB₅ toxin protein has an antigen attached thereto, and wherein the subject comprises cells that express a receptor for the AB₅ toxin protein.

128-130. (canceled)

131. The method of claim 127, wherein the antigen is a viral, bacterial, fungal, or parasite antigen, tumor-associated antigen, toxin antigen, or toxoid.

132-148. (canceled)
